(19) United States
(12) Patent Application Publication
(10) Pub. No.: US 2025/0082776 A1
(43) Pub. Date: Mar. 13, 2025

(54) POLYANION COATED LIPID NANOPARTICLES AND METHODS OF USE THEREOF

(71) Applicant: Massachusetts Institute of Technology, Cambridge, MA (US)

(72) Inventors: Paula T. Hammond-Cunningham, Newton, MA (US); Namita Rajesh Nabar, Cambridge, MA (US); Tamara Gomez Dacoba, Somerville, MA (US)

(73) Assignee: Massachusetts Institute Of Technology, Cambridge, MA (US)

(21) Appl. No.: 18/821,462

(22) Filed: Aug. 30, 2024

Related U.S. Application Data
(60) Provisional application No. 63/579,827, filed on Aug. 31, 2023.

(51) Int. Cl. A61K 47/69; A61K 9/127; A61K 9/51; A61K 31/7105; A61K 31/711

(52) U.S. Cl. CPC A61K 47/6913; A61K 9/1273; A61K 9/5123; A61K 31/7105; A61K 31/711

(57) ABSTRACT

Described herein are nanoparticles comprising an LNP core with electrostatically adsorbed anionic polymers layered on the LNP surface. These nanoparticles comprise varying nucleic acid cargos and may further comprise targeting moieties covalently attached to the anionic polymers. Also provided are methods of administering cargo to a subject, methods of treatment, methods of editing a gene, and methods of reducing non-targeted cell uptake of nanoparticles.

### CROSS-REFERENCE TO RELATED APPLICATIONS

This application claims priority under 35 U.S.C. § 119(e) to U.S. Provisional Patent Application, U.S. Ser. No. 63/579,827, filed Aug. 31, 2023, the contents of which is incorporated herein by reference in its entirety.

### BACKGROUND

Rapid advances in nucleic acid therapies highlight the immense therapeutic potential of genetic therapeutics. Lipid nanoparticles (LNPs) are highly potent non-viral transfection agents that can encapsulate and have demonstrated safe and efficacious delivery of various nucleic acid therapeutics, including but not limited to messenger RNA (mRNA), silencing RNA (siRNA), and plasmid DNA (pDNA). FDA-approved LNP therapies include Alnylam's Onpattro in 2018 (siRNA LNPs for hepatic transthyretin-mediated amyloidosis delivered intravenously) and Moderna and BioNTech/Pfizer's SARS-CoV-2 vaccines in 2020 (mRNA LNPs delivered intramuscularly). (1) However, a major challenge of targeted LNP-mediated systemic delivery is the nanoparticles' non-specific uptake by the liver and the mononuclear phagocytic system, due partly to the adsorption of endogenous serum proteins onto LNP surfaces (i.e., apolipoprotein E (ApoE) which binds low-density lipoprotein (LDL) receptors in hepatocytes). Tunable LNP surface chemistries may enable efficacious delivery across a range of organs and cell types.

Gene therapies, in the form of messenger RNA, small interfering RNA, or plasmid DNA, are promising treatments for genetic conditions. Lipid nanoparticles (LNPs) can effectively package and deliver these therapeutics. However, the efficacy of LNP-mediated gene delivery is challenged by off-target and non-specific uptake, by clearance organs such as the liver and by the mononuclear phagocytic system.

### SUMMARY

Disclosed herein are novel nanoparticles with modular surface chemistries that can be used to mitigate off-target and non-specific uptake while boosting transfection in target cells. The disclosed LNPs comprise a layer of biocompatible polyanions which are electrostatically layered onto the ionizable LNP core. Polyanion chemistries can be modified with a targeting moiety to induce specificity, allowing for targeted delivery of therapeutics.

In one aspect, provided herein is a nanoparticle, comprising:
(a) a lipid nanoparticle (LNP) core comprising an ionizable lipid and a cargo, wherein the ionizable lipid is positively charged; and
(b) an anionic polymer coating the LNP core; and wherein the anionic polymer is not nucleic acid-based.

In another aspect, provided is a pharmaceutical composition comprising a plurality of particles described herein, and an a pharmaceutically acceptable excipient.

In one aspect, the present disclosure provides a method of administering a cargo to a subject in need thereof, the method comprising administering to the subject a plurality of nanoparticles or the pharmaceutical composition described herein.

In another aspect, provided is a method of delivering a cargo to a target cell, the method comprising contacting the target cell with a nanoparticle or the pharmaceutical composition described herein.

In another aspect, provided herein is a method of treating a disease in a subject in need thereof, the method comprising administering to the subject a plurality of nanoparticles or the pharmaceutical composition described herein.

In one aspect, provided is a method of editing a gene in a cell, the method comprising contacting the cell the nanoparticle or the pharmaceutical composition described herein.

In another aspect, the present disclosure provides a method of reducing protein adsorption of a nanoparticle, the method comprising layering an anionic polymer on the outer surface of a nanoparticle comprising a therapeutic agent.

In one aspect, provided is a method of reducing non-targeted cell uptake of nanoparticles, the method comprising coating the nanoparticles with PAA.

In one aspect, provided herein is a kit comprising:
a plurality of nanoparticles or the pharmaceutical composition described herein; and
instructions for using the plurality of nanoparticles or the pharmaceutical composition.

### BRIEF DESCRIPTION OF THE DRAWINGS

FIG. 1A shows a schematic depicting a set of carboxylated polyanions with varying chemistries deposited onto positively charged LNP surfaces through electrostatic adsorption. FIG. 1B shows a schematic depicting process of layering LNPs with polyanions under stirring. FIG. 1C shows Z-averages measured via dynamic light scattering (DLS) of unlayered and layered mRNA-LNPs containing ALC-0315. N=3, 3 technical replicates. FIG. 1D shows surface zeta potential measurements of unlayered and layered mRNA-LNPs. N=3, 3 technical replicates; N=1, 3 technical replicates for UL. FIG. 1E shows mRNA encapsulation efficiency of unlayered and layered mRNA-LNPs. N=3. Ordinary one-way ANOVA, Tukey's test post hoc. ****p<0.0001, ***p<0.001, **p<0.01, *p<0.05. FIG. 1F shows representative negative-stained transmission electron micrography of unlayered and PAA-layered mRNA LNPs. All data represented as mean±SD.

FIG. 2A shows TNS titration curves of unlayered and layered mRNA-LNPs containing ALC-0315, curve visualized using locally weighted scatterplot smoothing. N=6 per reported value at a given pH; N=3 for HA-LLNPs. R² of fit=0.98, 0.96, 0.96, 0.94, 0.94 for UL, HA, PLD, PLE, and PAA respectively. FIG. 2B shows dynamic light scattering (DLS) measurements of z-averages of unlayered and layered LNPs after incubation in 100% mouse plasma. N=2 technical replicates. FIG. 2C shows fold change in total protein adsorbed onto LLNPs, relative to total protein adsorbed onto unlayered LNPs, as measured with a BCA assay. Data was fit to a second-order polynomial; R²=0.99. Standard curve was used to interpolate each sample value. N=3; 2 values in PAA-LLNP group were outside the interpolation range. Data represented as mean±SD. Ordinary one-way ANOVA, Tukey's test post hoc. ****p<0.0001.

FIG. 3A shows luciferase activity of HEK293T, RAW 264.7, EL4, and Jurkat cells treated with unlayered or layered luciferase mRNA-LNPs for 4 h, then incubated for 20 h. Data were normalized by cell viability; z-scores were plotted per cell line. N=3. FIG. 3B shows luciferase activity of cell lines treated with unlayered or layered luciferase mRNA-LNPs for 24 h. Data were normalized by cell viability; z-scores were plotted per cell line. N=3. FIG. 3C shows associated Cy5 signal of cell lines treated with unlayered or layered luciferase mRNA-LNPs for 4 h, then incubated for 20 h. Z-scores were plotted per cell line. N=3. FIG. 3D shows associated Cy5 signal of above-mentioned cell lines treated with unlayered or layered luciferase mRNA-LNPs for 24 h. Z-scores were plotted per cell line. N=3. FIG. 3E shows GFP-positive cell percentages of HEK293 Ts, EL4s, and HepG2s treated with unlayered or layered GFP-encoding pDNA LNPs for 4 h, then incubated for 20 h. N=3. Data represented as mean±SD. Ordinary one-way ANOVA, Tukey's test post hoc. ****p<0.0001, ***p<0.001, **p<0.01, *p<0.05.

FIG. 4 shows Confocal microscopy images of EL4 cells treated with unlayered or layered LNPs containing DOPE-Cy5, MFP488-labeled pDNA, and BDP-labeled polyanions (BODIPY=BDP). Cell membranes were visualized by staining cells with wheat germ agglutinin, AF405 conjugate. Images shown are representative z-slices. Imaged on Olympus FV1200, 100× magnification, 4 h after treatment. Size bars represent 10 microns.

FIG. 5A shows luciferase activity in liver, spleen, and lungs of C57BU6 mice 4 h after treatment with 0.3 mg/kg ALC-0315 mRNA-LLNPs. Flux was normalized by organ weight. N=5; N=4 for HA-LLNPs. FIG. 5B shows the ratio of weight-normalized luciferase signals in spleen and liver, 4 h after injection. N=5; N=4 for HA-LLNPs. FIG. 5C shows weight-normalized luciferase activity in liver, spleen, and lungs of C57BU6 mice 24 h after treatment with 0.3 mg/kg ALC-0315 mRNA-LLNPs. N=5. Data represented as mean±SD. Ordinary one-way ANOVA, Tukey's test post hoc. ****p<0.0001, ***p<0.001, **p<0.01, *p<0.05.

FIG. 6 shows SDS-PAGE depicting the range and molecular weights of adsorbed proteins on unlayered and layered Cy5-labeled LNPs containing ALC-0315. SDS-PAGE was run at 120V for 1 h 8 min.

FIGS. 7A-7B show mean fluorescence intensity (MFI) of (FIG. 7A) HepG2 liver carcinoma cells and (FIG. 7B) LOXIMVI melanoma cells, treated with unlayered or layered LNPs containing EGFP mRNA for 4 h, then washed and incubated in fresh media for 20 h. N=4. Ordinary one-way ANOVA, Tukey's test post hoc. ****p<0.0001, ***p<0.001, **p<0.01, *p<0.05. Data represented as mean±SD.

FIG. 8A shows luciferase activity of cell lines treated with unlayered or layered luciferase mRNA-LNPs for 4 h, then incubated for 20 h. Data was normalized by cell viability measured with PrestoBlue; z-scores were calculated within cell lines and plotted. N=3.

FIG. 8B shows luciferase activity of cell lines treated with unlayered or layered luciferase mRNA-LNPs for 24 h. Data was normalized by cell viability measured with PrestoBlue; z-scores were calculated within cell lines and plotted. N=3.

FIG. 9 shows confocal microscopy images of HEK293T cells treated with unlayered or layered LNPs containing DOPE-Cy5, MFP488-labeled pDNA, and BDP-labeled polyanions. Cell membranes were visualized by staining cells with wheat germ agglutinin, AF405 conjugate. Images shown are representative z-slices. Imaged on Olympus FV1200, 60× magnification, 4 h after treatment. Scale bars represent 25 microns.

FIG. 10A shows daily weight changes of C57BU6 mice dosed with 0.5 mg/kg luciferase-mRNA ALC-0315 LNPs, unlayered or layered with HA, PLD, PLE, or PAA. Weight changes expressed as percentage of weight at Day 0 prior to dosing, measured for 3 days. N=4; N=3 for PLE-LLNP group.

FIG. 10B shows enzyme levels of alkaline phosphatase (ALP), aspartate transaminase (AST), and alanine transaminase (ALT). N=4; N=3 for PLE-LLNP group.

FIG. 10C shows levels of blood urea nitrogen (BUN), creatinine (CREA), or total bilirubin in blood collected from C57BL/6 mice 72 h after being dosed with 0.5 mg/kg luciferase-mRNA LNPs, unlayered or layered. N=4; N=3 for PLE-LLNP group.

FIG. 10D shows levels of albumin and total protein in blood collected from C57BU/6 mice 72 h after being dosed with 0.5 mg/kg luciferase-mRNA LNPs, unlayered or layered. N=4; N=3 for PLE-LLNP group. Ordinary two-way ANOVA, Tukey's test post hoc. ****p<0.0001, ***p<0.001, **p<0.01, *p<0.05. Data represented as mean±SD.

FIG. 11 shows Luciferase activity in heart and kidneys of C57BU/6 mice 4 h after treatment with 0.3 mg/kg luciferase-mRNA ALC-0315 LNPs, unlayered or layered with HA, PLD, PLE, or PAA. Flux was normalized by mg organ tissue. N=5. Ordinary one-way ANOVA, Tukey's test post hoc. ****p<0.0001, ***p<0.001, **p<0.01, *p<0.05. Data represented as mean±SD.

FIG. 12 shows Luciferase activity in heart and kidneys of C57BU/6 mice 4 h after treatment with 0.3 mg/kg luciferase-mRNA ALC-0315 LNPs, unlayered or layered with HA, PLD, PLE, or PAA. Flux was normalized by mg organ tissue. N=5. Ordinary one-way ANOVA, Tukey's test post hoc. ****p<0.0001, ***p<0.001, **p<0.01, *p<0.05. Data represented as mean±SD.

FIG. 13A DOPE-Cy5 signal in serum of blood collected from C57BL/6 mice dosed with 0.3 mg/kg luciferase-RNA encoding unlayered or layered LNPs, doped with Cy5-DOPE. Cy5 signal was read on a Tecan plate reader and adjusted for background noise; log of signal was plotted for visualization. N=3.

FIG. 13B shows calculated half-life of each LNP, based on fit to one-phase decay; plateau set to 0.

FIG. 14 shows compositions of certain LNP formulations with varying nucleic acid cargo, component lipids, and lipid ratios.

FIG. 15 shows Z-average, PDI, and zeta potential of LNP formulations, both unlayered and layered with candidate polyanions, measured with DLS. N=3 technical replicates; N=9 technical replicates for Formulation 1 LLNPs. Data represented as mean±SD.

FIG. 16 shows Z-average, PDI, and zeta potential of unlayered and layered mRNA-LNPs containing DOPE-Cy5. N=3 technical replicates. Data represented as mean±SD.

FIG. 17 shows a summary schematic with sickled blood cells, example nanoparticles, and mouse model design.

FIGS. 18A-18B show LNP surface chemistry (FIG. 18A) and layer-by-layer LNP surface chemistry (FIG. 18B).

FIG. 19 shows a summary schematic of certain physical characterizations of LNPs.

FIGS. 20A-20D shows pDNA and mRNA encapsulation in unlayered LNPs. LNPs at identical compositions formed complexes with either pDNA or mRNA. FIG. 20A shows particle diameter (nm) to be 100-150 nm. FIG. 20B shows >+30 mV in surface potential. FIG. 20C shows >85% encapsulation of both therapeutic cargos was achieved. FIG. 20D shows representative image illustrates inverted micelle structure of LNPs encapsulating pDNA cargo. Image taken via negative-staining transmission electron microscopy.

FIG. 21A shows a schematic of governing parameters in pDNA LNP optimization in EL4 cells. FIG. 21B shows percent GFP positive cells after treatment with nanoparticles of varying molar compositions/ratios of cationic lipids, polyethylene glycol (PEG) lipids, phospholipids, and cholesterol. FIGS. 21C-21D show percent GFP positive cells after treatment with nanoparticles comprising varying cationic lipids and N/P ratios using the molar composition 3, n=3 biological replicates. Results were compared in a one-way ANOVA with post hoc Tukey's test. ****p<0.0001, **p<0.01, *p<0.05. Similar optimization was performed with mRNA LNPs in ER-HOXB8s.

FIG. 22A shows a schematic of a deposition of a set of carboxylated polyanions with varying chemistries (polysaccharide, polypeptide, or acrylic) onto the surfaces of cationic LNPs. FIG. 22B shows diameter and PDI of LNPs comprising varying polyanions. FIG. 22C shows zeta potential of LNPs comprising varying polyanions and loaded with pDNA (left) or mRNA (right). FIG. 22D shows pDNA encapsulation efficiency for LNPs comprising varying polyanions. FIGS. 22E-22F show electron microscope images of unlayered mRNA LNPs (FIG. 22E) and layered LNPs (FIG. 22F). Representative image shows LNPs layered with Polymer 4.

FIG. 23A shows percent GFP positive EL4 cells after 48 hr incubation with LNPs comprising varying polyanions. FIG. 23B shows percent GFP positive ER-HOXB8 cells after 24 hr incubation with LNPs comprising varying polyanions. Results were compared in a one-way ANOVA with post hoc Tukey's test. ****p<0.0001, **p<0.01, *p<0.05. All pairwise comparisons not indicated otherwise are significant (p<0.0001).

FIG. 24A shows an image of layered LNPs functionalized with HSC-targeting antibodies using click chemistry. FIG. 24B shows diameter and zeta potential of layered and functionalized LNPs. FIG. 24C depicts the experimental design of a C57Bl/6 mouse study. FIG. 24D shows percent Cy5 positive cells in T, B, and myeloid cell populations. Results were compared in a two-way ANOVA with post hoc Tukey's test. ****p<0.0001, **p<0.01, *p<0.05.

FIG. 25 shows a schematic of mouse studies with UL and LLNPs comprising mCherry mRNA and a DiD lipid fluorophore.

FIGS. 26A-26D show LNP accumulation and mCherry transfection for UL LNPs, PLD, HA, and PLE coated LLNPs 4- or 24-hours post-administration (UGT=urogenital tract).

FIG. 27A depicts a schematic of an PAA-azide layering of LNPs and conjugation of the antibody using click chemistry. FIG. 27B shows diameter (nm), polydispersity, and Zeta potential (mV) for UL LNPs, PAA LLNPs, and PAA LNPs with cKit (anti-CD117), anti-CD105, and non-targeting isotype antibodies on the surface. FIG. 27C shows electron micrographs of LLNPs.

FIG. 28A shows a schematic of Ai14 mice being administered varying LNPs by retroorbital injection and sacrificed on day 3 for FACS analysis. FIG. 28B shows the percent of whole bone marrow cells which are positive for tdTomato. FIG. 28C shows the percent of lineage negative (Lin-cKit+Scal+) cells which are positive for tdTomato. FIG. 28D shows the percent of long-term hematopoietic stem cells (LT-HSCs, LSK CD150+CD48−) which are positive for tdTomato. Grey triangle shows the decrease in differentiation between populations of cells. Results were compared in a one-way ANOVA with post hoc Tukey's test. ****p<0.0001, ***p<0.001, **p<0.01, *p<0.05, ns p>0.05.

FIG. 29A shows a schematic of an LLNP with an antibody appended to the surface as well as the Cas9 mRNA and gRNA cargo. FIG. 29B shows diameter (nm), polydispersity, and Zeta potential (mV) for two Cas9-loaded LLNPs (sgRosa26 and sgScramble (negative control)) layered with PAA and cKit or isotype antibodies.

FIG. 30A shows a schematic of the experimental design for Cas9-loaded LLNPs. FIG. 30B shows the percent insertions or deletions (indels) of the ROSA26 gene for Cas9 mRNA/sgRosa26 or the negative control Cas9 mRNA/sgScramble with UL LNPs, or PAA and cKit or isotype layered LLNPs.

### DEFINITIONS

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles (e.g., lipid nanoparticles), macroscopic particles, pellets, crystals, aggregates, composites, pulverized, milled or otherwise disrupted matrices, and cross-linked protein or polysaccharide particles. A particle may be composed of a single substance or multiple substances. In certain embodiments, the particle is a viral particle. In other embodiments, the particle is a liposome. In certain embodiments, the particle is a micelle. In certain embodiments, the particle is not a viral particle. In certain embodiments, the particle is not a micelle. In certain embodiments, the particle is substantially solid throughout. In certain embodiments, the particle is a nanoparticle. In certain embodiments, the particle is a microparticle. As used herein particle may refer to nanoparticle or lipid nanoparticle or LLNP.

The term “nanoparticle” refers to a particle having an average (e.g., mean) dimension (e.g., diameter) of between about 1 nanometer (nm) and about 1 micrometer (μm) (e.g., between about 1 nm and about 300 nm, between about 1 nm and about 100 nm, between about 1 nm and about 30 nm, between about 1 nm and about 10 nm, or between about 1 nm and about 3 nm), inclusive.

The term “microparticle” refers to a particle having an average (e.g., mean) dimension (e.g., diameter) of between about 1 micrometer (μm) and about 1 millimeter (mm) (e.g., between about 1 μm and about 100 μm, between about 1 μm and about 30 μm, between about 1 μm and about 10 μm, or between about 1 μm and about 3 μm), inclusive.

The term “polymer” refers to a compound comprising two or more covalently connected repeating units. In certain embodiments, a polymer is naturally occurring. In certain embodiments, a polymer is synthetic (i.e., not naturally occurring).

The term “polyelectrolyte” as used herein refers to a polymer which under a particular set of conditions (e.g., physiological conditions) has a net positive or negative charge. In some embodiments, a polyelectrolyte is or comprises a polycation; in some embodiments, a polyelectrolyte is or comprises a polyanion. Polycations have a net positive charge and polyanions have a net negative charge. The net charge of a given polyelectrolyte may depend on the surrounding chemical conditions, e.g., on the pH. Exemplary polyelectrolytes for use in polymeric coatings in the composition disclosed herein are but not limited to: poly(L-arginine) (PLR), poly-L-lysine (PLL), polyethylenimine (PEI), poly(β-amino esters), poly-L-glutamic acid (PLE), polyarginine, polyglutamic acid, polylysine, heparin folate, heparin sulfate, fucoidan, sulfated-β-cyclodextrin, hyaluronic acid (HA), polyglutamic acid-block-polyethylene glycol, poly-L-aspartic acid (PLD), polyaspartic acid, polystyrene sulfonate (SPS), polyacrylic acid (PAA), linear poly(ethylene imine) (LPEI), poly(diallyldimethyl ammonium chloride) (PDAC), polyallylamine hydrochloride (PAH), poly(L-lactide-co-L-lysine), polyserine ester, poly(4-hydroxy-L-proline ester), poly[α-(4-aminobutyl)-L-glycolic acid], sodium polystyrene sulfonate, dextran sulfate (DXS), alginate, and chondroitin sulfate.

The term “cation” or “cationic” used herein refer to a species which has a net positive charge. The term “anion” or “anionic” used herein refer to a species which has a net negative charge.

The term “sterol” refers to a subgroup of steroids also known as steroid alcohols, i.e., a steroid containing at least one hydroxyl group. Sterols are usually divided into two classes: (1) plant sterols also known as “phytosterols,” and (2) animal sterols also known as “zoosterols.” The term “sterol” includes, but is not limited to, cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers.

As used herein, the term “associated with” refers to a direct association between two molecules, due to, for example, covalent, electrostatic hydrophobic, and ionic and/or hydrogen-bond interactions.

As used herein, the term “affinity” refers to the equilibrium constant for the reversible binding of two agents (e.g., tag and a ligand, or a metal ion and a ligand) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of a ligand for unrelated peptides or compounds. Affinity of a ligand to its binding partner can be, for example, from about 100 nanomolar (nM) to about 0.1 nM, from about 100 nM to about 1 picomolar (pM), or from about 100 nM to about 1 femtomolar (fM) or more. An “affinity ligand” is a ligand having affinity for a binding partner.

Herein, the term “linker” (also known as “linker molecules” or “cross-linkers” or “spacers”) are molecules which may be used to conjugate one atom to another in a composition. The majority of known linkers react with amine, carboxyl, and sulfhydryl groups. Linker molecules may be responsible for different properties of the composition. The length of the linker should be considered in light of molecular flexibility during the conjugation step, and the availability of the conjugated molecule for its target. Longer linkers may improve the biological activity of the compositions as well as the ease of preparation of them. The geometry of the linker may be used to orient a molecule for optimal reaction with a target. A linker with flexible geometry may allow the entire composition to conformationally adapt as it binds a target sequence. The nature of the linker may be controlled by the monomeric units along with the polymer, e.g., a block polymer in which there is a block of hydrophobic monomers interspersed with a block of hydrophilic monomers.

The chemistry of preparing and utilizing a wide variety of molecular linkers is well-known in the art and many premade linkers for us in conjugating molecules are commercially available from vendors such as Pierce Chemical Co., Roche Molecular Biochemicals, United States Biological, VectorLabs, and BroadPharm.

Exemplary linker molecules for use in the compositions of the invention include, but are not limited to: maleimide-PEG4-DBCO, sulfo-maleimide-PEG4-DBCO, maleimide-PEG n-DBCO, sulfo maleimide-PEG n-DBCO, azido-PEG n-maleimide, or propargylamine, aminocaproic acid (ACA); polyglycine, and any other amino acid polymer polymers such as polyethylene glycol (PEG), polymethylmethacrylate (PMMA), polypropylene glycol (PPG); homobifunctional reagents such as APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS; heterobifunctional reagents such as ABH, AEDP, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, MBUS, M2C2H, MPBH, MSA, NHS-ASA, PDPH, PMPI, SADP, SAED SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, residues). Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS; and trifunctional linkers such as Sulfo-SBED. In certain embodiments, the linker comprises maleimide, PEG, and/or dibenzocyclooctyne (DBCO).

Branched linkers may be prepared or used so that multiple moieties per linker are able to react. Such multiply reactive linkers allow the creation of multimeric binding sites.

The term “pKa,” as used herein, includes the negative decadic logarithm of the ionization constant (Ka) of an acid; equal to the pH value at which equal concentrations of the acid and conjugate base forms of a substance (often a buffer) are present.

The term “hydrophobic,” as used herein, refers to a compound that has an octanol/water partition coefficient (K_{ow}) greater than about 10 at about 23° C.

The term “hydrophilic,” as used herein, refers to a compound that has an octanol/water partition coefficient (K_{ow}) less than about 10 at about 23° C.

As used here, the term “PEG-lipid” refers to a PEGylated lipid.

An “amino acid” refers to natural and unnatural D/L alpha-amino acids, as well as natural and unnatural beta- and gamma-amino acids. A “peptide” refers to two amino acids joined by a peptide bond. A “polypeptide” refers to three or more amino acids joined by peptide bonds. An “amino acid side chain” refers to the group(s) pended to the alpha carbon (if an alpha amino acid), alpha and beta carbon (if a beta amino acid), or the alpha, beta, and gamma carbon (if a gamma amino acid).

A “protein,” “peptide,” or “polypeptide” comprises a polymer of amino acid residues linked together by peptide bonds. The term, as used herein, refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

A cytokine is a signaling protein regulating biological functions such as innate and acquired immunity, hematopoiesis, inflammation and repair, and proliferation through mostly extracellular signaling. Interleukin 12 (I1-12 or IL-12) is a cytokine that is naturally produced by dendritic cells, macrophages, neutrophils, and human B-lymphoblastoid cells in response to antigenic stimulation. Il-12 is composed of a bundle of four alpha helices. It is a heterodimeric cytokine encoded by two separate genes, I1-12A and I1-12B. Single chain interleukin 12 (scIL-12) is a protein in which the subunits of heterodimeric IL-12 are covalently bonded together. For example, interleukin 12 can be monomerized by introduction of a peptide linker between the subunits of heterodimeric cytokine. scIL-12 may be a fusion protein.

The terms “composition” and “formulation” are used interchangeably.

A “target cell” refers to a cell in a subject (in vivo) or ex vivo to which a compound, particle, and/or composition of the present disclosure is delivered. A target cell may be an abnormal or unhealthy, which may need to be treated. A target cell may also be a normal or healthy but is under a higher-than-normal risk of becoming abnormal or unhealthy, which may need to be prevented. In certain embodiments, the target cell is a cancer cell. In certain embodiments, the target cell is an ovarian cancer cell (e.g., HM-1 cell). In certain embodiments, the target cell is a colon cancer cell. In certain embodiments, the target cell is a brain cancer cell. In certain embodiments, the target cell is a skin cancer cell. In certain embodiments, the target cell is a head and neck cancer cell. In certain embodiments, the target cell is a lung cancer cell.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the present disclosure is delivered. A target tissue may be an abnormal or unhealthy tissue, which may need to be treated. A target tissue may also be a normal or healthy tissue that is under a higher-than-normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a particle or plurality of particles described herein refers to an amount sufficient to elicit the desired biological response. An effective amount of a particle or plurality of particles described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a particle or plurality of particles described herein in a single dose. In certain embodiments, an effective amount is the combined amounts of a particle or plurality of particles described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a particle or plurality of particles for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a particle or plurality of particles per unit dosage form.

In certain embodiments, the particle or plurality of particles of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a particle or plurality of particles described herein is an amount sufficient to provide a therapeutic benefit in the treatment of a condition or to delay or minimize one or more symptoms associated with the condition. A therapeutically effective amount of a particle or plurality of particles means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a particle or plurality of particles described herein is an amount sufficient to prevent a condition, or one or more symptoms associated with the condition or prevent its recurrence. A prophylactically effective amount of a particle or plurality of particles means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The term “chemical handle” as used herein, refers to a functional group installed for further chemical modification. In some embodiments, the chemical handle comprised an azide, alkyne, amine, carboxylic acid, thiol, or other targetable moiety. In some embodiments, the chemical handle is maleimide. In some embodiments, the chemical handle comprises an alkyne or a cycloalkyne. In some embodiments, the chemical handle is reactive to click chemistry.

The phrase “hepatic uptake” as used herein described the specificity of nanoparticles (i.e., lipid nanoparticles) for uptake and/or degradation by the liver. LNPs preferentially accumulate in the liver, due in part to adsorption of serum proteins, that act as natural ligands for receptors on hepatocytes. In some embodiments, provided herein is a method of reducing hepatic uptake of nanoparticles by coating them with anionic polymers.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

The term “small molecule” refers to molecules, whether naturally-occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

The term “carbohydrate” or “saccharide” refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, disaccharides, or polysaccharides. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 2⁴) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an α anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, isomaltose, trehalose, cellobiose, xylobiose, laminaribiose, gentiobiose, mannobiose, melibiose, nigerose, or rutinose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein.

Anti-cancer agents encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g., HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), platinum containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., ‘2’-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AGO13736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleeveco, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

The term “gene” refers to a nucleic acid fragment that expresses a protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The terms “polynucleotide,” “nucleotide sequence,” “nucleic acid,” “nucleic acid molecule,” “nucleic acid sequence,” and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

The term “pDNA,” “plasmid DNA,” or “plasmid” refers to a small DNA molecule that is physically separate from, and can replicate independently of, chromosomal DNA within a cell. Plasmids can be found in all three major domains: Archaea, Bacteria, and Eukarya. In nature, plasmids carry genes that may benefit survival of the subject (e.g., antibiotic resistance) and can frequently be transmitted from one bacterium to another (even of another species) via horizontal gene transfer. Artificial plasmids are widely used as vectors in molecular cloning, serving to drive the replication of recombinant DNA sequences within host subjects. Plasmid sizes may vary from 1 to over 1,000 bp. Plasmids are considered replicons, capable of replicating autonomously within a suitable host.

The terms “nucleic acid” or “nucleic acid sequence,” “nucleic acid molecule,” “nucleic acid fragment,” or “polynucleotide” may be used interchangeably with “gene,” “mRNA encoded by a gene” and “cDNA.”

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLLUSLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

Immune disorders, such as auto-immune disorders, include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

Nucleic acid therapies have successfully been used to treat numerous genetic conditions and infectious diseases (1, 2). Modalities such as messenger RNA (mRNA), small interfering RNA (siRNA), and plasmid DNA (pDNA), can control levels of disease-relevant proteins via gene editing, silencing, or expression (2). Recent advances in gene editing, such as CRISPR/Cas systems, have significantly accelerated the development of curative therapies for hereditary disorders (3-6). However, translatable and scalable gene therapies require efficacious delivery carriers, since naked genetic vectors minimally penetrate cells and are rapidly degraded by enzymes in the body (7, 8).

Lipid nanoparticles (LNPs) are highly potent gene delivery vehicles that have been clinically translated in multiple applications, such as in vivo therapeutic delivery to the bone marrow, which expresses genetic diseases such as sickle cell disease and hemophilia. Patisiran, the first FDA-approved LNP therapy in 2018, delivers siRNA to the liver to treat transthyretin-mediated amyloidosis (1, 9). The 2020 emergency FDA approval and global deployment of the Moderna and Pfizer/BioNTech mRNA-LNP COVID-19 vaccines further demonstrated the translatability and therapeutic potential of LNP technology (1). LNPs are composed of four component lipids, each contributing to the nanocarrier's encapsulation and transfection potency: (a) Ionizable cationic lipids, which electrostatically bind anionic nucleic acids and drive transfection by inducing intracellular endosomal escape; (b, c) cholesterol and phospholipids, which contribute to structure and encapsulation; (d) lipid-anchored surface polyethylene glycol (PEG)-chains, which provide colloidal stability (7, 10).

When delivered intravenously, LNPs preferentially accumulate in the liver, due in part to adsorption of serum proteins, such as ApoE, that act as natural ligands for receptors on hepatocytes, and further facilitate rapid sequestration by immune cells (10, 11, 53). Liver accumulation is further aided by slow local blood flow, organ vasculature, and phagocytic cell phenotypes in hepatic sinusoids (12, 13). While these innate features greatly facilitate hepatic gene delivery, they pose a challenge for the many genetic disorders that require extrahepatic delivery (11, 14).

Certain therapeutic applications require well-defined LNP selectivity as well as efficacy in order to avoid off-target effects or optimize treatments. Selectivity can be gained by controlling surface protein adsorption and introducing more specific targeting interactions. PEG-functionalized lipids ensure serum stability of LNPs, but may elicit immune responses in chronic or repeat LNP doses, due to anti-PEG antibodies in humans (11). Furthermore, PEGylated lipids exist in constant equilibrium between their free form and their incorporated state in the LNP; their constant lipid exchange allows for additional serum protein adsorption (11). Prior work has illustrated that the chemistries of the four component lipids can be tuned to drive LNP potency and biodistribution. Combinatorial libraries of ionizable lipid chemistries have identified structures capable of redirecting maximal LNP transfection from the liver to target areas, such as the lungs or spleen (15-17). Other studies have demonstrated the impact of ionizable lipid chemistry and charged helper lipids on redirecting LNP biodistribution and transfection, by tuning the size, surface charge, and pKa of LNPs (18, 19). Although these parameters can be used to leverage the protein corona for targeted delivery where advantageous, identification of hit formulations requires the synthesis and screening of hundreds of lipid structures, is not straightforward, does not yield predictable results, and has failed to date to produce successful results.

Without wishing to be bound by theory, the inventors hypothesized that LNP trafficking, transfection, and biodistribution could be tuned with Layer-by-Layer (LbL) self-assembly.

Provided herein are novel formulations of layered LNPs (LLNPs) that comprise charged bioactive polymers deposited onto the LNP surfaces. This layering acts as a tunable modality to alter the NP's surface presentation and cellular interactions both in vitro and in vivo, and can be generalized to facilitate incorporation of other therapeutic or targeting moieties into LNP delivery systems. Varying polyanion chemistries can modulate selectivity in LNP uptake and transfection, helping to address the ongoing challenge of targeted nucleic acid delivery. Indeed, an optimized LNP delivery vector may benefit significantly from the combination of a highly efficacious ionizable lipid to boost transfection and a rationally chosen surface polyanion to provide on-target selectivity.

Unexpectedly, layering of LNPs with the bioinert polymer, PAA, resulted in decreased uptake and transfection across cell lines. These properties make PAA a desirable “stealth” layer to minimize off-target uptake, for cases in which an additional targeting moiety is utilized.

HSPCs are a high-potential therapeutic target, however, non-viral transfection of these cells is challenged by the facts that (1) native HSPCs reside in a quiescent state outside the cell cycle, and are therefore resistant to particle uptake and transfection, and (2) in vivo therapies are more likely to be taken up by other, more populous hematopoietic populations in the peripheral blood, including myeloid and lymphoid cells. The disclosed LLNPs further comprise a targeting moiety address these two challenges.

To achieve stable adsorption, LNPs were titrated to a positive charge, then incubated in solutions of polyanions. The inventors unexpectedly discovered that layering was generalizable across cores; stable LLNPs were formed from a diverse set of LNP cores varying in component lipids and nucleic acid cargos. Tested polyanions included biologically relevant chemistries, such as polysaccharides, homopolypeptides, and synthetic hydrocarbon backbones (21, 22). The inventors discovered that surface adsorption of a polyanion alters the physicochemical properties of LNPs while maintaining encapsulation of nucleic acid cargos. Rational choice of polyanion modulated cell-specific uptake and transfection in vitro and in vivo to improve NP targeting specificity. This method of stable electrostatic adsorption onto LNP surfaces provides a simple and modular platform to control LNP targeting and specificity, and allows incorporation of more diverse targeting and therapeutic molecules to benefit on-target nucleic acid delivery.

The LLNPs disclosed herein can be purified by dialysis, ultracentrifugation, or tangential flow filtration. To further enable surface functionalization, polyanion carboxylate groups can be modified with any of a family of linker groups, including but not limited to azide, propargyl, and tetrazine groups, to enable covalent attachment of functionalized targeting moieties, including but not limited to antibodies and antibody fragments, peptides, and nanobodies, modified with reactive linker pairs, including but not limited to DBCO, azide, and TCO groups.

In one aspect, provided herein is a nanoparticle, comprising:
(a) a lipid nanoparticle (LNP) core comprising an ionizable lipid and a cargo, wherein the ionizable lipid is positively charged; and
(b) an anionic polymer coating the LNP core; and
   wherein the anionic polymer is not nucleic acid-based.

In some embodiments, the LNP core comprises: a sterol; a polyethylene glycol (PEG) lipid; a phospholipid; and an ionizable lipid. In certain embodiments, the LNP core is positively charged. In some embodiments, the LNP core comprises a positively charged outer surface In some embodiments, the LNP core is not layered with a nucleic acid cargo. In some embodiments, the LNP core does not comprise a positively charged polymer or amino acid (i.e., poly-arginine). In some embodiments, the LNP core does not comprise poly-arginine.

In certain embodiments, the sterol is cholesterol.

In some embodiments, the PEG lipid is a PEG-ceramide lipid, DMG-PEG-2000, DOPE-PEG-2000, or DOPE-2000-N3. In some embodiments, the PEG lipid is DMG-PEG-2000. In some embodiments, the PEG lipid is a PEG-ceramide lipid. In some embodiments, the PEG lipid is DOPE-PEG-2000. In some embodiments, the PEG lipid is DOPE-PEG2000-N₃. In some embodiments, the PEG lipid is modified with a chemical handle.

In certain embodiments, the phospholipid is selected from 1,2-dioleoyl-sn-glycero-3-phosphoethanolamine (DOPE), 1,2-distearoyl-sn-glycero-3-phosphocholine (DSPC), and 1,2-dioleoyl-sn-glycero-3-phosphocholine (DOPC). In certain embodiments, the phospholipid is DOPE-PEG2000-N₃. In certain embodiments, the phospholipid is DOPE. In certain embodiments, the phospholipid is DSPC. In certain embodiments, the phospholipid is DOPC. In some embodiments, the phospholipid is modified with a chemical handle.

In some embodiments, the ionizable lipid is selected from ALC-0315, DLin-MC3-DMA, DLin-KC2-DMA, cKK-E12, C12-200, and SM-102. In some embodiments, the ionizable lipid is selected from ALC-0315, DLin-MC3-DMA, DLin-KC2-DMA, cKK-E12, C12-200, SM-102, 306-012B, or dioleoyl-3-trimethylammonium propane (DOTAP). In some embodiments, the ionizable lipid is ALC-0315. In some embodiments, the ionizable lipid is DLin-MC3-DMA. In some embodiments, the ionizable lipid is DLin-KC2-DMA. In some embodiments, the ionizable lipid is cKK-E12. In some embodiments, the ionizable lipid is C12-200. In some embodiments, the ionizable lipid is SM-102. In some embodiments, the ionizable lipid is 306-012B. In some embodiments, the ionizable lipid is DOTAP.

In some embodiments, the ionizable lipid comprises ALC-0315. In some embodiments, the ionizable lipid comprises DLin-MC3-DMA. In some embodiments, the ionizable lipid comprises DLin-KC2-DMA. In some embodiments, the ionizable lipid comprises cKK-E12. In some embodiments, the ionizable lipid comprises C12-200. In some embodiments, the ionizable lipid comprises SM-102. In some embodiments, the ionizable lipid comprises 306-012B. In some embodiments, the ionizable lipid comprises DOTAP. In some embodiments, the ionizable lipid comprises a chemical handle.

In certain embodiments, the anionic polymer is carboxylated or sulfonated. In some embodiments, the anionic polymer comprises sulfonate, carboxylate, or phosphonate moieties. In certain embodiments, the anionic polymer is carboxylated. In certain embodiments, the anionic polymer is sulfonated. In certain embodiments, the anionic polymer is an oligosaccharide (i.e. glucan). In certain embodiments, the anionic polymer is a protein. In certain embodiments, the anionic polymer is a homoprotein. In certain embodiments, the anionic polymer is a peptide. In certain embodiments, the anionic polymer is a homopeptide. In certain embodiments, the anionic polymer is synthetic. In some embodiments, the anionic polymer comprises a chemical handle.

In some embodiments, the anionic polymer comprises hyaluronic acid (HA), poly-L-glutamate (PLE), poly-L-aspartate (PLD), polyacrylic acid (PAA), dextran sulfate, chondroitin sulfate, fucoidan, heparin sulfate, alginate, pegylated-poly-L-glutamic acid, pegylated-poly-L-aspartic acid, polysialic acid, carboxymethyl cellulose, methacrylate, or sulfated polybeta cyclodextrin. In some embodiments, the anionic polymer comprises at least two different polymers. In some embodiments, the anionic polymer is a single polymer. In some embodiments, the anionic polymer is hyaluronic acid (HA), poly-L-glutamate (PLE), poly-L-aspartate (PLD), polyacrylic acid (PAA), dextran sulfate, chondroitin sulfate, fucoidan, heparin sulfate, alginate, pegylated-poly-L-glutamic acid, pegylated-poly-L-aspartic acid, polysialic acid, carboxymethyl cellulose, methacrylate, or sulfated polybeta cyclodextrin.

In some embodiments, the anionic polymer is dextran sulfate. In some embodiments, the anionic polymer is chondroitin sulfate. In some embodiments, the anionic polymer is fucoidan. In some embodiments, the anionic polymer is heparin sulfate. In some embodiments, the anionic polymer is alginate. In some embodiments, the anionic polymer is pegylated-poly-L-glutamic acid. In some embodiments, the anionic polymer is pegylated-poly-L-aspartic acid. In some embodiments, the anionic polymer is polysialic acid. In some embodiments, the anionic polymer is carboxymethyl cellulose. In some embodiments, the anionic polymer is methacrylate. In some embodiments, the anionic polymer is sulfated polybeta cyclodextrin. In some embodiments, the anionic polymer chemically modified to comprise a clickable handle (i.e. azide, alkyne etc.).

In certain embodiments, the anionic polymer comprises HA, PLD, PLE, or PAA. In certain embodiments, the anionic polymer comprises HA. In certain embodiments, the anionic polymer comprises PLD. In certain embodiments, the anionic polymer comprises PLE. In certain embodiments, the anionic polymer comprises PAA.

In some embodiments, wherein the anionic polymer is HA, PLD, PLE, or PAA. In some embodiments, wherein the anionic polymer is HA. In some embodiments, wherein the anionic polymer is PLD. In some embodiments, wherein the anionic polymer is PLE. In some embodiments, wherein the anionic polymer is PAA.

In some embodiments, the anionic polymer is not DNA or RNA. In some embodiments, the anionic polymer is not mRNA. In some embodiments, the particle does not comprise a poly-cationic polymer layer between the core and the anionic polymer. In some embodiments, the particle does not comprise a poly-arginine polymer layer between the core and the anionic polymer. In some embodiments, the LNP core does not comprise a poly-cationic polymer. In some embodiments, the LNP core does not comprise a poly-arginine.

In some embodiments, the anionic polymer is 5-120 kDa in molecular weight. In some embodiments, the anionic polymer is 10-20 kDa in molecular weight. In some embodiments, the anionic polymer is about 10-20 kDa in molecular weight. In some embodiments, the anionic polymer is about 10 kDa in molecular weight. In some embodiments, the anionic polymer is about 15 kDa in molecular weight. In some embodiments, the anionic polymer is about 20 kDa in molecular weight.

In certain embodiments, the nanoparticle is about 50-300 nm in size. In certain embodiments, the nanoparticle is 50-300 nm in size. In certain embodiments, the nanoparticle is 50-100 nm in size. In certain embodiments, the nanoparticle is 100-150 nm in size. In certain embodiments, the nanoparticle is 150-200 nm in size. In certain embodiments, the nanoparticle is 200-250 nm in size. In certain embodiments, the nanoparticle is 250-300 nm in size. In certain embodiments, the nanoparticle is about 100 nm in size. In certain embodiments, the nanoparticle is about 200 nm in size. In certain embodiments, the nanoparticle is about 100 nm in size. In certain embodiments, the nanoparticle is about 110 nm in size. In certain embodiments, the nanoparticle is about 120 nm in size. In certain embodiments, the nanoparticle is about 130 nm in size. In certain embodiments, the nanoparticle is about 140 nm in size. In certain embodiments, the nanoparticle is about 150 nm in size.

In some embodiments, the nanoparticle is about 100-200 nm in size.

In certain embodiments, the cargo is a nucleic acid. In certain embodiments, the cargo is a pharmaceutical agent (i.e, small molecule, protein, peptide). In some embodiments, there is more than one cargo. In some embodiments, the cargo comprises a protein. In some embodiments, the cargo comprises a complex of guide RNA and ribonucleoprotein (RNP). In some embodiments, the cargo comprises a cationic nuclear localization peptide covalently or electrostatically bound to pDNA. In some embodiments, the cargo is inside the LNP core. In some embodiments, the cargo is not layered around the core.

In some embodiments, the nucleic acid is DNA or RNA.

In some embodiments, the nucleic acid is DNA. In certain embodiments, the cargo is plasmid DNA (pDNA). In certain embodiments, the cargo is single-stranded DNA (ssDNA), double-stranded DNA (dsDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA.

In certain embodiments, the nucleic acid is RNA (mRNA), plasmid DNA (pDNA), small interfering RNA (siRNA), small guide RNA (sgRNA).

In certain embodiments, the nucleic acid is RNA. In certain embodiments, the cargo is RNA. In some embodiments, the RNA is coding RNA or non-coding RNA. In some embodiments, the coding RNA is messenger RNA (mRNA). In some embodiments, the RNA is precursor messenger RNA. In some embodiments, the non-coding RNA is double-stranded RNA, short hairpin RNA, microRNA, guide RNA, transfer RNA, antisense RNA, long non-coding RNA, signal recognition particle RNA, small cytoplasmic RNA, small nuclear RNA, ribosomal RNA, Piwi-interacting RNA, small nucleolar RNA, or spliced leader RNA. In some embodiments, the non-coding RNA is small interfering RNA. In some embodiments, the RNA is single-stranded RNA, heterogeneous nuclear RNA, satellite RNA, viral RNA, or viral satellite RNA. In some embodiments, the RNA is single guide RNA (sgRNA). In some embodiments, the RNA is prime editing guide RNA (pegRNA). In some embodiments, the RNA is a ribonucleoprotein (RNP) complex. In some embodiments, the RNP complex comprises Cas9 protein and sgRNA. In some embodiments, the agent is ribozyme or flexizyme. In some embodiments, the polynucleotide is a DNA. In some embodiments, the DNA is a plasmid DNA (pDNA).

In certain embodiments, the cargo is small interfering RNA (siRNA). In certain embodiments, the cargo is messenger RNA (mRNA). In certain embodiments, the cargo is single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, the agent is an RNA that carries out RNA interference (RNAi). The phenomenon of RNAi is discussed in greater detail, for example, in the following references: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553. In certain embodiments, upon delivery of an RNA into a subject, tissue, or cell, the RNA is able to interfere with the expression of a specific gene in the subject, tissue, or cell. In certain embodiments, the agent is a pDNA, siRNA, mRNA, or a combination thereof.

In some embodiments, the nucleic acid is siRNA, miRNA, gRNA, mRNA, dsRNA, shRNA, or asRNA. In certain embodiments, the nucleic acid is gRNA. In some embodiments, the nucleic acid is mRNA. In some embodiments, the cargo is Cas9 mRNA.

In certain embodiments, the nanoparticle further comprises a targeting moiety. In some embodiments, the targeting moiety is a protein, peptide, saccharide, or nucleic acid. In certain embodiments, the targeting moiety is selected from an antibody, nanobody, affibody, aptamer, cyclodextrin, or a derivative or fragment thereof. In certain embodiments, the targeting moiety is an antibody. In certain embodiments, the targeting moiety is nanobody. In certain embodiments, the targeting moiety is affibody. In certain embodiments, the targeting moiety is aptamer. In certain embodiments, the targeting moiety is cyclodextrin. In certain embodiments, the targeting moiety is an scFv.

In some embodiments, the targeting moiety targets stem cells, immune cell, or cancer cells. In some embodiments, the targeting moiety targets healthy cells. In some embodiments, the targeting moiety targets immune cells. In some embodiments, the targeting moiety targets white blood cells, leukocytes, or B-cells. In some embodiments, the targeting moiety targets cancer cells. In certain embodiments, the targeting moiety targets stem cells. In some embodiments, the stem cells are hematopoietic progenitor stem cells (HSPC). In certain embodiments, the targeting moiety targets cancer cells. In some embodiments, the cancer cells are ovarian cancer cells or blood cancer cells. In certain embodiments, the targeting moiety targets ovarian tumor cells. In certain embodiments, the targeting moiety targets blood cancer cells. In certain embodiments, the targeting moiety targets sickle blood cells.

In certain embodiments, the targeting moiety is an antibody. In some embodiments, the antibody is anti-CD117, anti-CD105, anti-CD90, anti-CXCR4, anti-CD45, anti-CD4, anti-CD8, anti-CD3, anti-CD19, anti-CD20, or anti-ferritin antibody. In certain embodiments, the targeting moiety is an anti-CD117 antibody. In certain embodiments, the targeting moiety is an anti-CD105 antibody. In certain embodiments, the targeting moiety is an anti-CD90 antibody. In certain embodiments, the targeting moiety is an anti-CXCR4. In certain embodiments, the targeting moiety is an anti-CD45 antibody. In certain embodiments, the targeting moiety is an anti-CD4 antibody anti-CD8. In certain embodiments, the targeting moiety is an anti-CD3 antibody. In certain embodiments, the targeting moiety is an anti-CD19 antibody. In certain embodiments, the targeting moiety is an anti-CD20 antibody. In certain embodiments, the targeting moiety is an anti-ferritin antibody. In some embodiments, the targeting moiety is anti-CD117, anti-CD105, anti-CD90, anti-CXCR4, anti-CD45, anti-CD4, anti-CD8, anti-CD3, anti-CD19, anti-CD20, or anti-ferritin scFv.

In some embodiments, the targeting moiety is anti-CD117, anti-CD105, anti-CD90, anti-CXCR4, anti-CD45, anti-CD4, anti-CD8, anti-CD3, anti-CD19, anti-CD20, or anti-ferritin nanobody.

In certain embodiments, the targeting moiety is a peptide. In some embodiments, the peptide is SV40 T antigen NLS, M9 NLS, AP2, or RAP12. In some embodiments, the targeting moiety is SV40 T antigen NLS. In some embodiments, the targeting moiety is M9 NLS. In some embodiments, the targeting moiety is AP2. In some embodiments, the targeting moiety is RAP12.

In certain embodiments, the targeting moiety is a nanobody. In some embodiments, the nanobody is an anti-CD117 nanobody, anti-CD45 nanobody, anti-CD105 nanobody, anti-CD90 nanobody, anti-CXCR4 nanobody, anti-CD4 nanobody, anti-CD8 nanobody, anti-CD3 nanobody, anti-CD19 nanobody, anti-CD20 nanobody, or anti-ferritin nanobody.

In certain embodiments, the targeting moiety is covalently bound or electrostatically associated to the nanoparticle. In certain embodiments, the targeting moiety is covalently bound to the nanoparticle core. In certain embodiments, the targeting moiety is electrostatically associated with the nanoparticle core.

In some embodiments, the targeting moiety is covalently bound to the anionic polymer. In certain embodiments, the targeting moiety is covalently bound to the anionic polymer by click chemistry. In certain embodiments, the targeting moiety is covalently bound to the LNP core by click chemistry.

In certain embodiments, the linker comprises maleimide, PEG, and dibenzocyclooctyne (DBCO). In certain embodiments, the linker comprises maleimide. In certain embodiments, the linker comprises PEG. In certain embodiments, the linker comprises DBCO.

In some embodiments, the targeting moiety is bound to the anionic polymer by a linker. In some embodiments, the linker comprises a chemical handle (i.e., clickable handle (azide, alkyne etc.)) In some embodiments, the linker comprises maleimide-PEG4-DBCO chemistry, sulfo-maleimide-PEG4-DBCO chemistry, maleimide-PEG n-DBCO chemistry, sulfo maleimide-PEG n-DBCO chemistry, azido-PEG n-maleimide chemistry, or propargylamine chemistry.

In some embodiments, the targeting moiety is covalently bound to PAA. In some embodiments, the targeting moiety is covalently bound to PLE. In some embodiments, the targeting moiety is covalently bound to HA. In some embodiments, the targeting moiety is covalently bound to PLD.

In some embodiments, the nanoparticle comprises:
a positively charged lipid nanoparticle core comprising cholesterol, DMG-PEG-2000, DOPE, and cKK-E12;
wherein the LNP core is electrostatically coated with PAA; and
wherein the nanoparticle further comprises anti-CD117.

In some embodiments, the nanoparticle comprises:
a positively charged lipid nanoparticle core comprising cholesterol, a PEG lipid selected from: PEG-ceramide lipid, DMG-PEG-2000, DOPE-PEG-2000, or DOPE-2000-N₃, a phospholipid selected from DOPC, DSPC, and DOPE, and an ionizable lipid selected from ALC-0315, DLin-MC3-DMA, DLin-KC2-DMA, cKK-E12, C12-200, 306-012B, SM-102, and DOTAP;
wherein the LNP core is electrostatically coated with a the polyanion PAA, HA, PLD, PAA, dextran sulfate, chondroitin sulfate, fucoidan, heparin sulfate, alginate, pegylated-poly-L-glutamic acid, pegylated-poly-L-aspartic acid, polysialic acid, carboxymethyl cellulose, methacrylate, or sulfated polybeta cyclodextrin; and
wherein the nanoparticle further comprises an antibody selected from an anti-CD117, anti-CD105, anti-CD90, anti-CXCR4, anti-CD45, anti-CD4, anti-CD8, anti-CD3, anti-CD19, anti-CD20, and anti-ferritin antibody; and a nucleic acid cargo.

In certain embodiments, the LNP core comprises 40% cKK-E12, 16% DOPE, 1% DMG-PEG2000, and 43% cholesterol. In some embodiments, the LNP core comprises 30-60% ionizable lipid. In some embodiments, the LNP core comprises 30-40% ionizable lipid. In some embodiments, the LNP core comprises 40-50% ionizable lipid. In some embodiments, the LNP core comprises 50-60% ionizable lipid. In some embodiments, the LNP core comprises about 40% of ionizable lipid.

In some embodiments, the LNP core comprises 5-30% of phospholipid. In some embodiments, the LNP core comprises 5-15% of phospholipid. In some embodiments, the LNP core comprises 15-20% of phospholipid. In some embodiments, the LNP core comprises 15-30% of phospholipid. In some embodiments, the LNP core comprises about 15% of phospholipid. In some embodiments, the LNP core comprises 16% of phospholipid.

In some embodiments, the LNP core comprises 0.1-5% of PEG lipid. In some embodiments, the LNP core comprises 1% of PEG lipid. In some embodiments, the LNP core comprises 2% of PEG lipid. In some embodiments, the LNP core comprises 3% of PEG lipid. In some embodiments, the LNP core comprises 4% of PEG lipid. In some embodiments, the LNP core comprises 5% of PEG lipid.

In some embodiments, the LNP core comprises 20-75% sterol. In some embodiments, the LNP core comprises 20-30% sterol. In some embodiments, the LNP core comprises 30-45% sterol. In some embodiments, the LNP core comprises 40-50% sterol. In some embodiments, the LNP core comprises 50-75% sterol. In some embodiments, the LNP core comprises 60-75% sterol. In some embodiments, the LNP core comprises about 45% sterol. In some embodiments, the LNP core comprises 43% sterol.

In another aspect, provided is a pharmaceutical composition comprising a plurality of particles described herein, and a pharmaceutically acceptable excipient. In certain embodiments, the pharmaceutical composition further comprises an additional pharmaceutical agent. In some embodiments, the additional pharmaceutical agent is selected from the group consisting of a chemotherapeutic agent, targeted therapy, gene therapy, immune therapy, and hormone therapy.

The present disclosure provides pharmaceutical compositions comprising a plurality of particles described herein, and optionally a pharmaceutically acceptable excipient. In certain embodiments, the pharmaceutical composition described herein comprises a plurality of LNPs described herein, and a pharmaceutically acceptable excipient.

In certain embodiments, the particle or plurality of particles described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., proliferative disease, hematological disease, immune disorder, neurological disease, painful condition, psychiatric disorder, or metabolic disorder) in a subject in need thereof.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present ex vivo.

Pharmaceutical compositions described herein can be prepared by any method known in the art of pharmaceutics. In general, such preparatory methods include bringing the particle or plurality of particles described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents or fillers, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents or fillers include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, starches (such as dry starch, cornstarch), sugars (such as powdered sugar), calcium trisulfate, carboxymethylcellulose calcium, dextrate, dextrin, dextrose, fructose, lactitol, lactose, magnesium carbonate, magnesium, maltitol, maltodextrin, maltose, sucrose, glucose, mannitol, silicic acid, xylitol, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary disintegrating agents or disintegrants include agar, algin, alginic acid, sodium alginate, silicates, sodium carbonate, calcium carbonate, carboxymethylcellulose, cellulose, clay, colloidal silicon dioxide, croscarmellose sodium, crospovidone, rubber, magnesium silicate, methylcellulose, potassium krillin, hydroxypropylcellulose (e.g., low substituted Hydroxypropylcellulose), crosslinked polyvinylpyrrolidone, hydroxypropylcellulose, and starch (e.g., sodium glycolate starch, potato or tapioca starch).

Exemplary binding agents include starch (e.g., glycolate starch, cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include agar, ethyl oleate, ethyl laurate, glycerin, blyceryl palmitostearate, magnesium oxide, magnesium stearate, mannitol, poloxamer, glycol, sodium stearyl, sorbitol, zinc stearate, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, camauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

In some embodiments, injectable preparations of the compositions disclosed herein are in the form of a ready-to-use (“RTU”) preparation that can be directly administered to a subject. In some embodiments, the RTU preparation is a suspension. In some embodiments, the RTU preparation is a solution. In some embodiments, the RTU preparation is an emulsion. In some embodiments, injectable preparations of the compositions disclosed herein are in the form of a solid that is reconstituted prior to administration. In some embodiments, the solid is a lyophilized solid. In some embodiments, injectable preparations of the compositions disclosed herein are in the form of a liquid or suspension that is diluted prior to administration.

In some embodiments, the pharmaceutical compositions disclosed herein comprise a bulking agent. Bulking agents can be used, e.g., to improve the appearance of a solid composition, to provide visible “bulk” to demonstrate product quality or to facilitate preparation, e.g., of a solid composition prepared for reconstitution prior to administration. Bulking agents can be used for low dose (high potency) drugs that do not have the necessary bulk to support their own structure or provide a visible composition in a unit dosage form. Bulking agents are used in lyophilized formulations. Bulking agents provide a desirable structure for a lyophilized cake comprising pores that provide the means for vapor to escape from the product during lyophilization cycles, and facilitate dissolution on reconstitution. In some embodiments, the bulking agent is mannitol, lactose, sucrose, dextran, trehalose, povidone, dextran, glycine, isoleucine, methionine, or a cyclodextrin (e.g., (2-hydroxypropyl)-β-cyclodextrin).

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The particle or plurality of particles provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The particle or plurality of particles, as described herein, can be administered in combination with one or more additional pharmaceutical agents (e.g., therapeutically and/or prophylactically active agents). The particle or plurality of particles, or compositions, can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a particle or plurality of particles described herein and an additional pharmaceutical agent shows a synergistic effect that is absent in a pharmaceutical composition including one of the particle or plurality of particles and the additional pharmaceutical agent, but not both. In some embodiments, the additional pharmaceutical agent achieves a desired effect for the same disorder. In some embodiments, the additional pharmaceutical agent achieves different effects.

The particle or plurality of particles, or composition, can be administered concurrently with, prior to, or subsequent to one or more additional pharmaceutical agents, which may be useful as, e.g., combination therapies. Pharmaceutical agents include therapeutically active agents. As used herein, therapeutic agent and pharmaceutical agent are used interchangeably. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In one aspect, the present disclosure provides a method of administering a cargo to a subject in need thereof, the method comprising administering to the subject a plurality of nanoparticles or the pharmaceutical composition described herein.

In another aspect, provided is a method of delivering a cargo to a target cell, the method comprising contacting the target cell with a nanoparticle or the pharmaceutical composition described herein.

In certain embodiments, the cargo is a nucleic acid. In certain embodiments, the cargo is a pharmaceutical agent (i.e., small molecule, protein, peptide).

In some embodiments, the nucleic acid is DNA or RNA.

In some embodiments, the nucleic acid is DNA. In certain embodiments, the cargo is plasmid DNA (pDNA). In certain embodiments, the cargo is single-stranded DNA (ssDNA), double-stranded DNA (dsDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA.

In certain embodiments, the nucleic acid is RNA (mRNA), plasmid DNA (pDNA), small interfering RNA (siRNA), small guide RNA (sgRNA).

In certain embodiments, the nucleic acid is RNA. In certain embodiments, the cargo is RNA. In some embodiments, the RNA is coding RNA or non-coding RNA. In some embodiments, the coding RNA is messenger RNA (mRNA). In some embodiments, the RNA is precursor messenger RNA. In some embodiments, the non-coding RNA is double-stranded RNA, short hairpin RNA, microRNA, guide RNA, transfer RNA, antisense RNA, long non-coding RNA, signal recognition particle RNA, small cytoplasmic RNA, small nuclear RNA, ribosomal RNA, Piwi-interacting RNA, small nucleolar RNA, or spliced leader RNA. In some embodiments, the non-coding RNA is small interfering RNA. In some embodiments, the RNA is single-stranded RNA, heterogeneous nuclear RNA, satellite RNA, viral RNA, or viral satellite RNA. In some embodiments, the RNA is single guide RNA (sgRNA). In some embodiments, the RNA is prime editing guide RNA (pegRNA). In some embodiments, the RNA is a ribonucleoprotein (RNP) complex. In some embodiments, the RNP complex comprises Cas9 protein and sgRNA. In some embodiments, the agent is ribozyme or flexizyme. In some embodiments, the polynucleotide is a DNA. In some embodiments, the DNA is a plasmid DNA (pDNA).

In certain embodiments, the cargo is small interfering RNA (siRNA). In certain embodiments, the cargo is messenger RNA (mRNA). In certain embodiments, the cargo is single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, the agent is an RNA that carries out RNA interference (RNAi). The phenomenon of RNAi is discussed in greater detail, for example, in the following references: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553. In certain embodiments, upon delivery of an RNA into a subject, tissue, or cell, the RNA is able to interfere with the expression of a specific gene in the subject, tissue, or cell. In certain embodiments, the agent is a pDNA, siRNA, mRNA, or a combination thereof.

In some embodiments, the nucleic acid is siRNA, miRNA, gRNA, mRNA, dsRNA, shRNA, or asRNA. In certain embodiments, the nucleic acid is gRNA. In some embodiments, the nucleic acid is mRNA. In some embodiments, the cargo is Cas9 mRNA. In certain embodiments, the RNA is siRNA, miRNA, gRNA, mRNA, dsRNA, shRNA, or asRNA.

In some embodiments, the target cell is an immune cell, a hematopoietic cell, a stem cell, or a cancer cell. In some embodiments, the target cell is an immune cell (i.e., B-cell, lymphocyte, leukocyte, etc.). In some embodiments, the target cell is a hematopoietic cell. In some embodiments, the target cell is a stem cell. In some embodiments, the target cell is a hematopoietic stem cell (i.e. HSPC). In some embodiments, the target cell is a cancer cell. In some embodiments, the target cell is an ovarian cancer cell. In some embodiments, the target cell is a blood cancer cell. In some embodiments, the target cell is a tumor cell. In some embodiments, the target cell is a healthy cell. In some embodiments, the target cell is not diseased.

In certain embodiments, the target cell is a CD117-presenting cell, CD105-presenting cell, CD90-presenting cell, CXCR4-presenting cell, CD45-presenting cell, CD4-presenting cell, CD8-presenting cell, CD3-presenting cell, CD19-presenting cell, CD20-presenting cell, or ferritin-presenting cell. In certain embodiments, the target cell is a CD117-presenting cell. In certain embodiments, the target cell is a CD105-presenting cell. In certain embodiments, the target cell is a CD90-presenting cell. In certain embodiments, the target cell is a CXCR4-presenting cell. In certain embodiments, the target cell is a CD45-presenting cell. In certain embodiments, the target cell is a CD4-presenting cell. In certain embodiments, the target cell is a CD8-presenting cell. In certain embodiments, the target cell is a CD3-presenting cell. In certain embodiments, the target cell is a CD19-presenting cell. In certain embodiments, the target cell is a CD20-presenting cell. In certain embodiments, the target cell is a ferritin-presenting cell.

In another aspect, provided herein is a method of treating a disease in a subject in need thereof, the method comprising administering to the subject a plurality of nanoparticles or the pharmaceutical composition described herein.

In some embodiments, the method further comprises administering to the subject an additional pharmaceutical agent.

In certain embodiments, the disease is a proliferative disease, an immune disorder, or genetic disease. In some embodiments, the proliferative disease is cancer. In certain embodiments, the cancer is ovarian or blood cancer. In certain embodiments, the cancer is ovarian cancer. In certain embodiments, the cancer is blood cancer. In some embodiments, the disease is genetic disease. In some embodiments, the genetic disease is sickle cell anemia. In some embodiments, the disease is an immune disease. In some embodiments, the disease is an autoimmune disorder.

In one aspect, provided is a method of editing a gene in a cell, the method comprising contacting the cell the nanoparticle or the pharmaceutical composition described herein.

In some embodiments the cell is an immune cell, a hematopoietic cell, a stem cell, or a cancer cell. In some embodiments, the target cell is an immune cell, a hematopoietic cell, a stem cell, or a cancer cell. In some embodiments, the cell is an immune cell (i.e., B-cell, lymphocyte, leukocyte, etc.). In some embodiments, the cell is a hematopoietic cell. In some embodiments, the cell is a stem cell. In some embodiments, the cell is a hematopoietic stem cell (i.e. HSPC). In some embodiments, the cell is a cancer cell. In some embodiments, the cell is an ovarian cancer cell. In some embodiments, the cell is a blood cancer cell. In some embodiments, the cell is a tumor cell. In some embodiments, the cell is a healthy cell. In some embodiments, the cell is not a diseased cell.

In certain embodiments, the cell is a CD117-presenting cell, CD105-presenting cell, CD90-presenting cell, CXCR4-presenting cell, CD45-presenting cell, CD4-presenting cell, CD8-presenting cell, CD3-presenting cell, CD19-presenting cell, CD20-presenting cell, or ferritin-presenting cell.

In certain embodiments, the cell is a CD117-presenting cell, CD105-presenting cell, CD90-presenting cell, CXCR4-presenting cell, CD45-presenting cell, CD4-presenting cell, CD8-presenting cell, CD3-presenting cell, CD19-presenting cell, CD20-presenting cell, or ferritin-presenting cell. In certain embodiments, the cell is a CD117-presenting cell. In certain embodiments, the cell is a CD105-presenting cell. In certain embodiments, the cell is a CD90-presenting cell. In certain embodiments, the cell is a CXCR4-presenting cell. In certain embodiments, the cell is a CD45-presenting cell. In certain embodiments, the cell is a CD4-presenting cell. In certain embodiments, the cell is a CD8-presenting cell. In certain embodiments, the cell is a CD3-presenting cell. In certain embodiments, the cell is a CD19-presenting cell. In certain embodiments, the cell is a CD20-presenting cell. In certain embodiments, the cell is a ferritin-presenting cell.

In certain embodiments, the cell is a stem cell. In some embodiments, the stem cell is a hematopoietic stem cell.

In another aspect, the present disclosure provides a method of reducing protein adsorption of a nanoparticle, the method comprising layering an anionic polymer on the outer surface of a nanoparticle comprising a therapeutic agent. Therapeutic agent as used herein related to any compound or composition with pharmacological relevance. In some embodiments, the therapeutic agent is an anti-cancer agent. In some embodiments, provided is a method of reducing protein adsorption of a nanoparticle, the method comprising layering an anionic polymer on the outer surface of lipid nanoparticles described herein.

In certain embodiments, the method modulates hepatic uptake. In some embodiments, the method reduces hepatic uptake. In certain embodiments, the anionic polymer is HA, PLE, PLD, PAA, dextran sulfate, chondroitin sulfate, fucoidan, heparin sulfate, alginate, pegylated-poly-L-glutamic acid, polysialic acid, carboxymethyl cellulose, methacrylate, sulfated polybeta cyclodextrin. In certain embodiments, the anionic polymer is HA. In certain embodiments, the anionic polymer is PLE. In certain embodiments, the anionic polymer is PLD. In certain embodiments, the anionic polymer is PAA. In certain embodiments, the anionic polymer is dextran sulfate. In certain embodiments, the anionic polymer is chondroitin sulfate. In certain embodiments, the anionic polymer is fucoidan. In certain embodiments, the anionic polymer is heparin sulfate. In certain embodiments, the anionic polymer is alginate. In certain embodiments, the anionic polymer is pegylated-poly-L-glutamic acid. In certain embodiments, the anionic polymer is polysialic acid. In certain embodiments, the anionic polymer is carboxymethyl cellulose. In certain embodiments, the anionic polymer is methacrylate. In certain embodiments, the anionic polymer is sulfated polybeta cyclodextrin. In certain embodiments, the anionic polymer is not HA.

In one aspect, provided is a method of reducing non-targeted cell uptake of nanoparticles, the method comprising coating the nanoparticles with PAA. In certain embodiments, the method further comprises the addition of a targeting moiety. In certain embodiments, the method further comprises the addition of another polymer. In certain embodiments, the method further comprises the addition of a cationic polymer layer. In certain embodiments, the method further comprises the addition of an additional anionic polymer. In some embodiments, the anionic polymer comprises at least two different polymers.

In some embodiments, the targeting moiety is a protein, peptide, saccharide, or nucleic acid.

In certain embodiments, the targeting moiety is selected from an antibody, nanobody, affibody, aptamer, cyclodextrin, or a derivative or fragment thereof. In certain embodiments, the targeting moiety is selected from an antibody, nanobody, affibody, aptamer, cyclodextrin, or a derivative or fragment thereof. In certain embodiments, the targeting moiety is an antibody. In certain embodiments, the targeting moiety is nanobody. In certain embodiments, the targeting moiety is affibody. In certain embodiments, the targeting moiety is aptamer. In certain embodiments, the targeting moiety is cyclodextrin. In certain embodiments, the targeting moiety is an scFv.

In some embodiments, the targeting moiety targets stem cells, immune cell, or cancer cells. In some embodiments, the targeting moiety targets healthy cells. In some embodiments, the targeting moiety targets immune cells. In some embodiments, the targeting moiety targets white blood cells, leukocytes, or B-cells. In some embodiments, the targeting moiety targets cancer cells. In certain embodiments, the targeting moiety targets stem cells. In some embodiments, the stem cells are hematopoietic progenitor stem cells (HSPC). In certain embodiments, the targeting moiety targets cancer cells. In some embodiments, the cancer cells are ovarian cancer cells or blood cancer cells. In certain embodiments, the targeting moiety targets ovarian tumor cells. In certain embodiments, the targeting moiety targets blood cancer cells. In certain embodiments, the targeting moiety targets sickle blood cells.

In certain embodiments, the targeting moiety is an antibody. In some embodiments, the antibody is anti-CD117, anti-CD105, anti-CD90, anti-CXCR4, anti-CD45, anti-CD4, anti-CD8, anti-CD3, anti-CD19, anti-CD20, or anti-ferritin antibody. In certain embodiments, the targeting moiety is an anti-CD117 antibody. In certain embodiments, the targeting moiety is an anti-CD105 antibody. In certain embodiments, the targeting moiety is an anti-CD90 antibody. In certain embodiments, the targeting moiety is an anti-CXCR4. In certain embodiments, the targeting moiety is an anti-CD45 antibody. In certain embodiments, the targeting moiety is an anti-CD4 antibody anti-CD8. In certain embodiments, the targeting moiety is an anti-CD3 antibody. In certain embodiments, the targeting moiety is an anti-CD19 antibody. In certain embodiments, the targeting moiety is an anti-CD20 antibody. In certain embodiments, the targeting moiety is an anti-ferritin antibody. In some embodiments, the targeting moiety is anti-CD117, anti-CD105, anti-CD90, anti-CXCR4, anti-CD45, anti-CD4, anti-CD8, anti-CD3, anti-CD19, anti-CD20, or anti-ferritin scFv. In certain embodiments, the antibody is anti-CD117, anti-CD105, anti-CD90, anti-CXCR4, anti-CD45, anti-CD4, anti-CD8, anti-CD3, anti-CD19, anti-CD20, or anti-ferritin antibody.

In one aspect, provided is a method of making a LNP comprising an outer polyelectrolyte layer, the method comprising: (1) providing an LNP core and, (2) contacting the LNP core with the polyelectrolyte under stirred conditions. In some embodiments, the stirred conditions comprise mechanical stirring. In some embodiments, the mechanical stirring is in the range of 300 RPM to 2000 RPM. In some embodiments, the mechanical stirring is in the range of 700 RPM to 800 RPM. In some embodiments, the mechanical stirring is in the range of 300 RPM to 600 RPM. In some embodiments, the mechanical stirring is in the range of 600 RPM to 800 RPM. In some embodiments, the mechanical stirring is in the range of 800 RPM to 1000 RPM. In some embodiments, the mechanical stirring is in the range of 1000 RPM to 1200 RPM. In some embodiments, the mechanical stirring is in the range of 1200 RPM to 1400 RPM. In some embodiments, the mechanical stirring is in the range of 1400 RPM to 1600 RPM. In some embodiments, the mechanical stirring is in the range of 1600 RPM to 1800 RPM. In some embodiments, the mechanical stirring is in the range of 1800 RPM to 2000 RPM. In some embodiments, the mechanical stirring is in the range of 2000 RPM to 2200 RPM. In some embodiments, the mechanical stirring is in the range of 2200 RPM to 2400 RPM. In some embodiments, the mechanical stirring is in the range of 2400 RPM to 2600 RPM. In some embodiments, the mechanical stirring is in the range of 2600 RPM to 2800 RPM. In some embodiments, the mechanical stirring is in the range of 2800 RPM to 3000 RPM. In some embodiments, the stirred conditions comprise microfluidic mixing. In some embodiments, the microfluidic mixing is 1-20 mL/min. In some embodiments, the microfluidic mixing is 1-5 mL/min. In some embodiments, the microfluidic mixing is 5-10 mL/min. In some embodiments, the microfluidic mixing is 10-15 mL/min. In some embodiments, the microfluidic mixing is 15-20 mL/min.

In one aspect, provided herein is a kit comprising:
a plurality of nanoparticles or the pharmaceutical composition described herein; and
instructions for using the plurality of nanoparticles or the pharmaceutical composition.

The kits provided may comprise a pharmaceutical composition or particle or plurality of particles described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a particle or plurality of particles or pharmaceutical composition described herein. In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. A kit described herein may include one or more additional pharmaceutical agents described herein as a separate composition.

### EXAMPLES

### Example 1: Electrostatic Adsorption of Polyanions onto Lipid Nanoparticles Controls Uptake, Trafficking, and Transfection of RNA and DNA Therapies

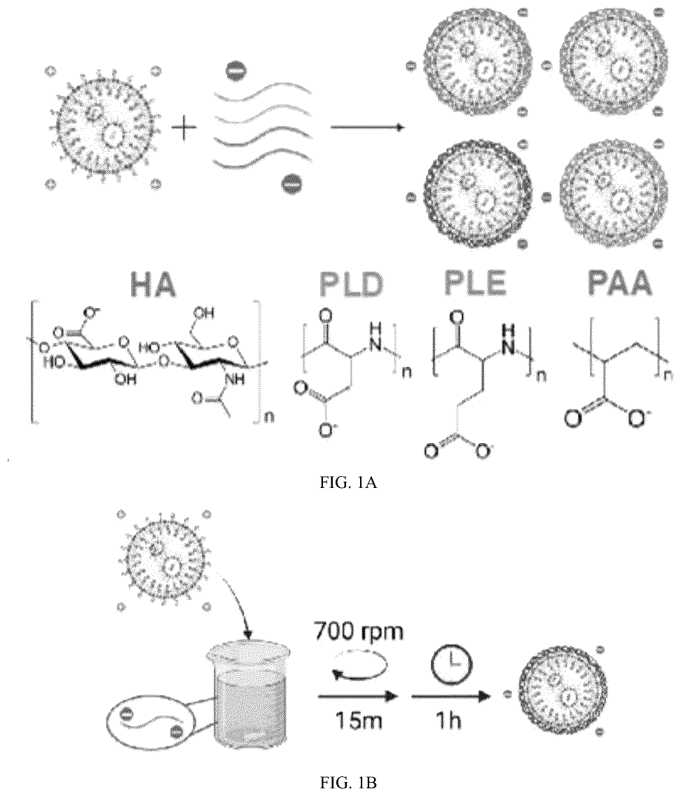

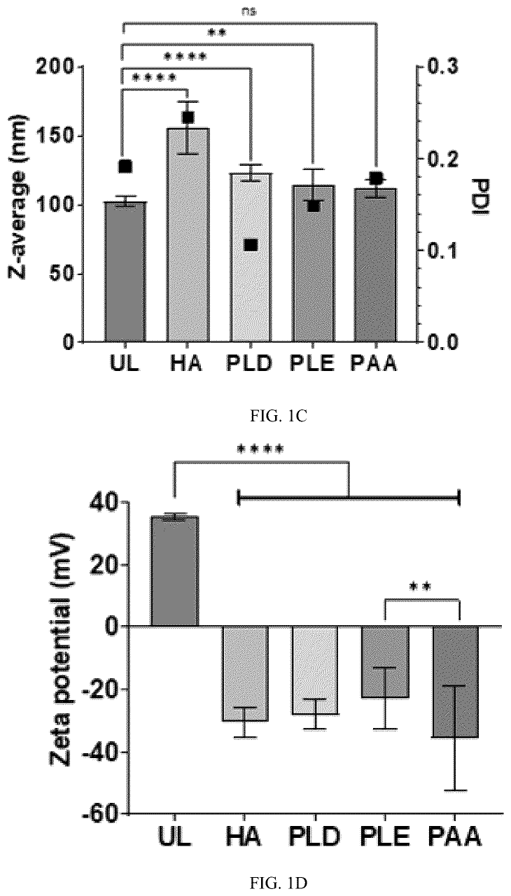

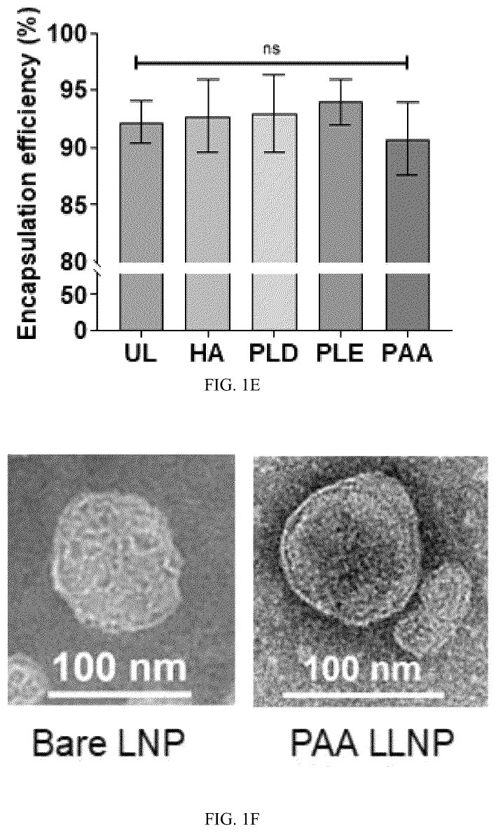

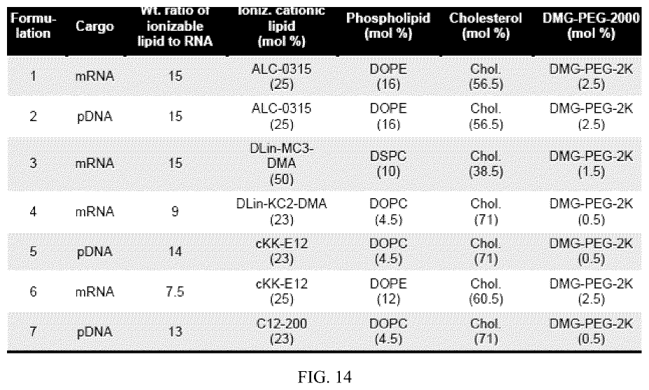

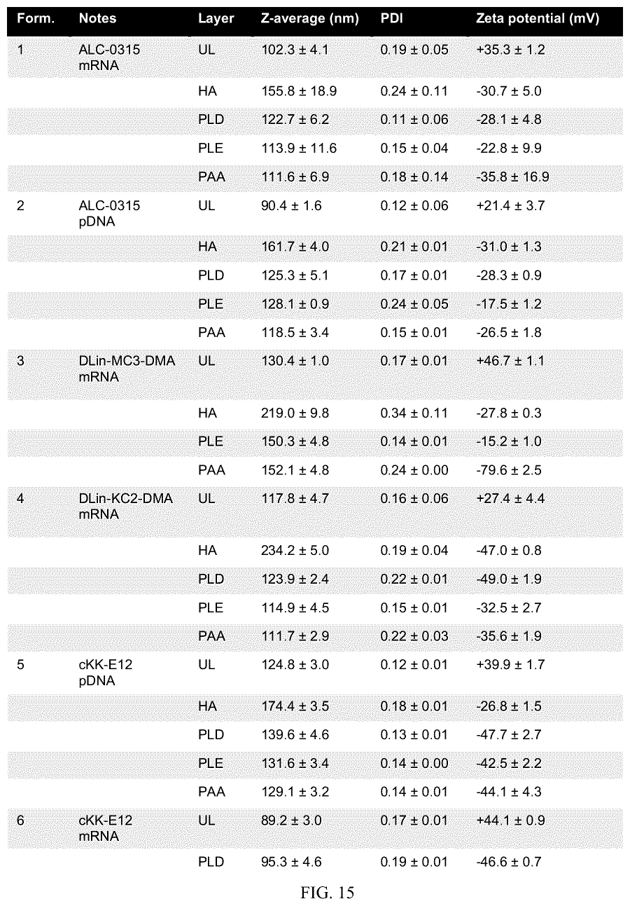

LNPs can be stably layered with biologically relevant polyanions. To test the impact of polyanion structure on the performance of LLNPs, LNPs were layered with a library of four carboxylated polyanions: hyaluronate (HA), poly-L-aspartate (PLD), poly-L-glutamate (PLE), and poly-acrylate (PAA) (FIG. 1A). These biopolymers were chosen for their previously demonstrated specificity for cancer cell lines and primary cell types (16, 17, 20). The polyanions varied in monomer structures, sampling biologically relevant saccharides (HA), uniformly repeating poly(amino acid)s (PLD, PLE), and hydrocarbon-based acrylate backbones (PAA). Polyanion molecular weights ranged between 14 and 20 kDa. To test whether layering was modular across LNPs, a set of LNP cores were formulated with varying in component lipids, lipid ratios, and nucleic acid cargos (either mRNA or pDNA). Formulations contained commercially available ionizable cationic lipids used in prior applications, with either single valency (DLin-MC3-DMA, DLin-KC2-DMA, ALC-0315) or multiple valency (C12-200, cKK-E12) (FIG. 14) (2, 27-30). LNPs were suspended in acidic conditions to express positive surface charge, then incubated with equal volumes of each of the four polyanions (FIG. 1B). Polyanions are between 10-20 kDa and dissolved in aqueous buffer. Stable layering was defined as a state achieving complete surface charge conversion from >30 mV to <−30 mV, with diameters below 200 nm as measured by dynamic light scattering (DLS). Relative to unlayered LNPs, LLNP diameters increased by 5 to 50 nm (FIGS. 1C, 15). Variations in sizes are attributed to both polyelectrolyte secondary interactions and hydration effects. For example, HA, which caused the largest diameter increases, can strongly self-associate through hydrogen bonding, leading to a thicker adsorbed layer; its swelling may also be due to high levels of water association (31, 32). Layering LNPs induced complete charge reversal, from unlayered LNP zeta potentials of +35±1 mV to layered zeta potentials of −23±10 to −36±17 mV (FIG. 1D). The magnitude of charge reversal depended on the weight equivalents of polyanion used for layering. Nucleic acid encapsulation efficiency did not change significantly upon addition of surface layers, suggesting that the polyanions do not displace encapsulated cargo (FIG. 1E). Furthermore, LLNPs retain internal core structure, as confirmed by transmission electron micrography (TEM) (FIG. 1F).

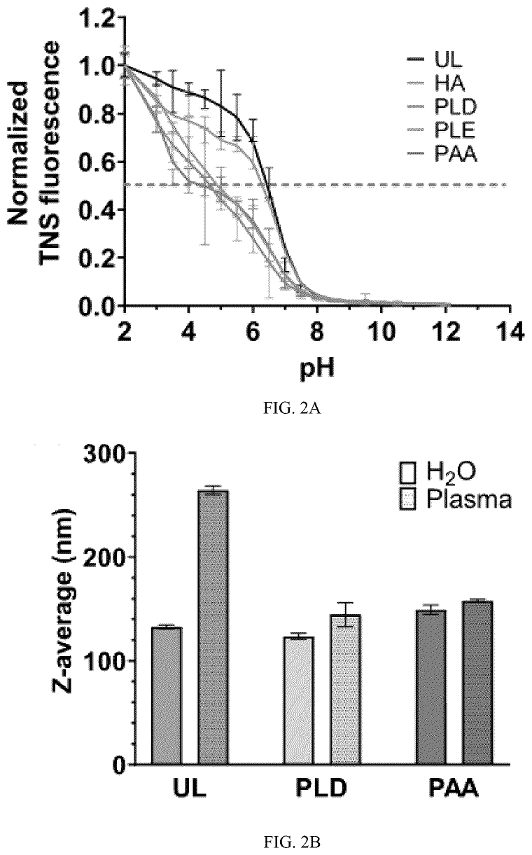

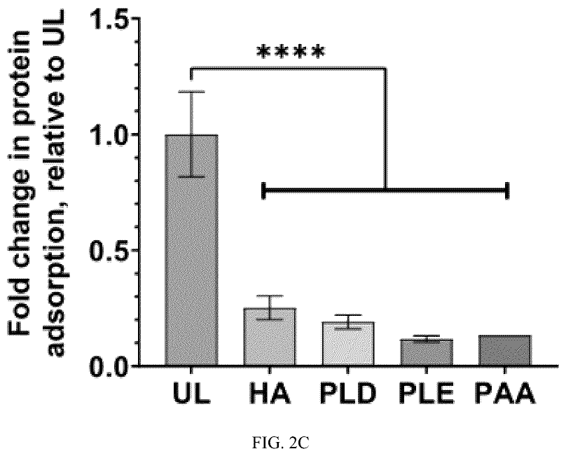

Surface polyanion alters apparent pKa of LNPs. Without wishing to be bound by theory, the inventors hypothesized that the addition of a charged polyanion with acidic carboxylic groups changes the apparent pKa of the LLNP. Prior work optimizing ionizable lipids has demonstrated that the apparent pKa of the LNP impacts its transfection potency, immunogenicity, and targeting in vivo (18, 28, 33-35). The impact of surface polyanions on the apparent pKa of LLNPs was therefore quantified. To illustrate trends in pKa, mRNA-LNPs with ionizable lipid ALC-0315, which has a reported pKa of 6.5, were layered with the four polyanions. ALC-0315 was chosen as a representative lipid given its monovalency and use in the Pfizer/BioNTech COVID-19 vaccine (1). Apparent pKa was determined using a four-parameter logistic regression of 2-p-toluidinonaphthalene-6-sulfonic acid (TNS), an anionic fluorophore that emits signal when in hydrophobic environments, such as cation through previously established binding assays (35). Briefly, pKa was reported as the pH at which the normalized TNS fluorescence, a proxy for LNP protonation, was 50% of the maximum detected TNS fluorescence. While unlayered LNPs exhibited a pKa of 6.5 (95% CI[6.4, 6.6]), the addition of PLD, PLE, and PAA decreased the apparent pKa to 4.7 (95% CI[4.4, 4.9]), 5.0 (95% CI[4.7, 5.3]), and 4.4 (95% CI[3.3, 4.9]) respectively (FIG. 2A). The acidified pKa of LLNPs suggest that the deposited polyanion's functional groups—in this case, acidified carboxyl groups—can modulate the pKa of the LNP (FIG. 2A). Notably, HA-LLNPs exhibited a pKa of 6.5 (95% CI[6.3, 6.6]) similar to unlayered LNPs, though HA itself has a reported pKa ranging from 3 to 4 (36, 37). Furthermore, the pKa curves of LLNPs showed a buffering zone, within which TNS fluorescence plateaued, between pH 3.5-4, 3.5-4.5, and 3.5-5 for PLD, PLE, and PAA, respectively. The presence of two ionizable species—the polyanion and the ionizable lipid—may create a multi-step equilibrium.

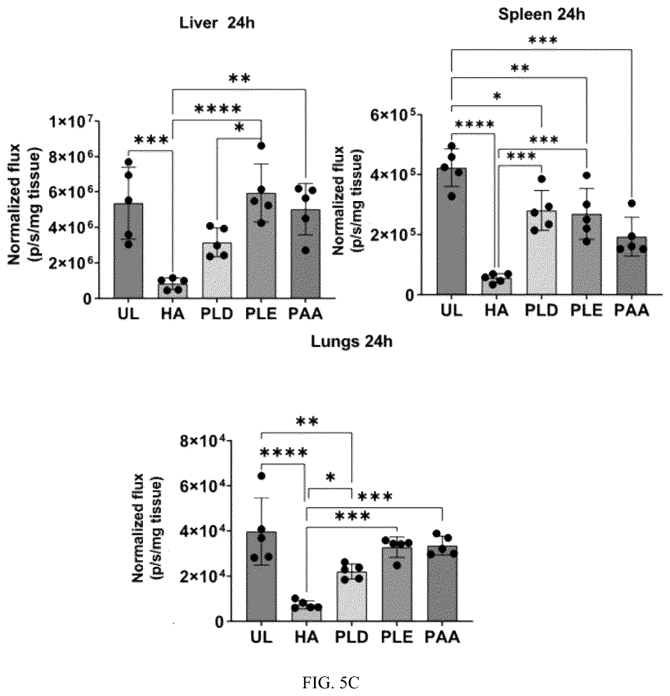

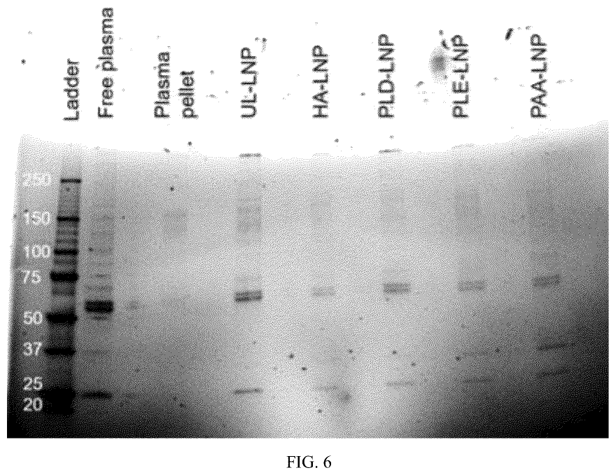

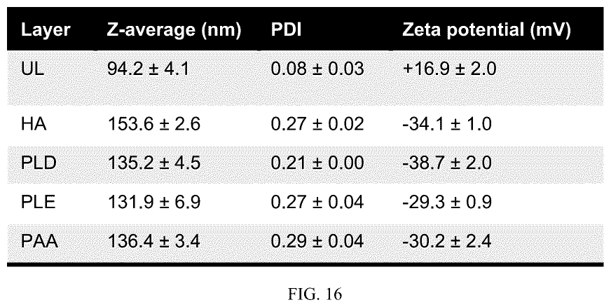

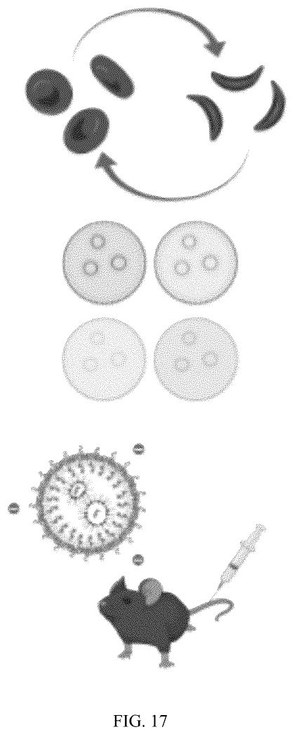

Surface polyanion reduces LNP surface protein adsorption. Given that LNP extrahepatic delivery is hindered by adsorption of liver-targeting endogenous proteins, the impact of the surface polyanion on protein adsorption was quantified (10). To test the impact of the surface polyanion on LNP stability in biological conditions, both unlayered and LLNPs were incubated in either mouse plasma or water at 37° C., then washed via centrifugation. DLS measurements indicated that unlayered LNPs in mouse plasma were 131±6 nm larger than LNPs in water, likely due to the adsorption of plasma proteins. By contrast, PLD-LLNPs displayed a size increase of 21±15 nm; PAA-LLNPs displayed a size increase of 9±6 nm (FIG. 2B). This improved stability may be attributed to the polyanion's ability to mitigate protein adsorption. In a separate study, unlayered and layered LNPs containing DOPE-Cy5 were similarly incubated in mouse plasma for 1 h at 37° C. and washed via centrifugation. Total adsorbed plasma protein content was quantified with a BCA assay, a standard curve of baseline-corrected values was normalized by Cy5 signal. Within this study, LLNPs adsorbed four-fold less total plasma protein than did unlayered LNPs (FIG. 2C). The zeta potentials of all LLNPs tested were between −29.3±0.9 and −38.7±2.0 mV (FIG. 16); therefore, differences in adsorbed protein content are not attributed to any major differences in surface charge. Differences in adsorbed protein profiles have been shown to influence biodistribution and transfection in vivo (38). Preliminary characterization of adsorbed protein profiles was visualized with SDS-PAGE (FIG. 6), however, further proteomics analysis of the proteins preferentially bound to each layer will provide greater insights into LLNPs' cellular interactions in future work.

Surface polyanion alters LNP transfection of mRNA and pDNA. To quantify the impact of LNP surface chemistry on transfection, different cell lines were treated with unlayered and layered LNPs containing luciferase-encoding mRNA. To isolate the impact of the polyanion, LNP core formulations were held constant in a 25/52.5/16/2.5 ratio of ALC-0315/cholesterol/DOPE/DMG-PEG-2000, then layered with each polyanion. Cell lines included HEK293T, adherent epithelial cells; RAW 264.7, highly phagocytic macrophages; and EL4 and Jurkats, T lymphoma cells with low uptake rates. To assess the impact of the surface layer on rates of uptake and internalization, cells were treated with LNPs for either 4 h or 24 h. HEK293T cells were dosed at 50 ng mRNA/well; all other cell lines were dosed at 100 ng mRNA/well. For the shorter treatment, cells were washed at 4 h and incubated for an additional 20 h in fresh media before readout, to allow time for detectable protein translation.

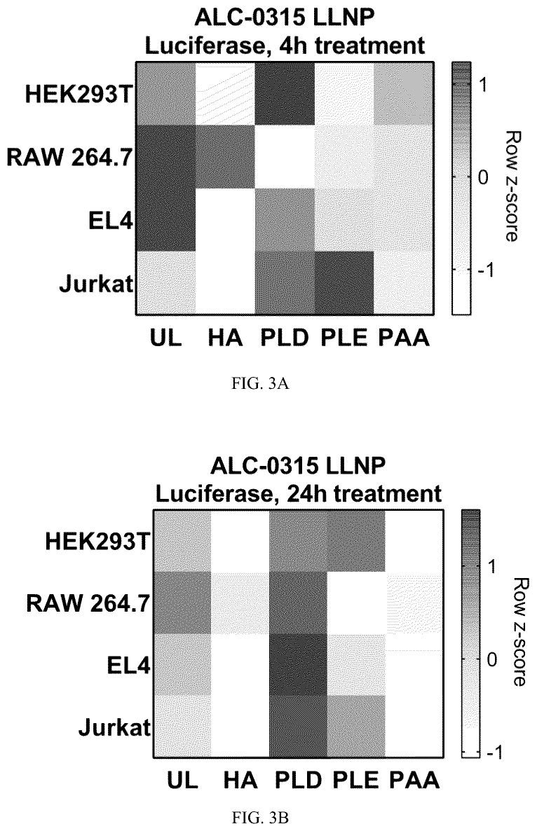

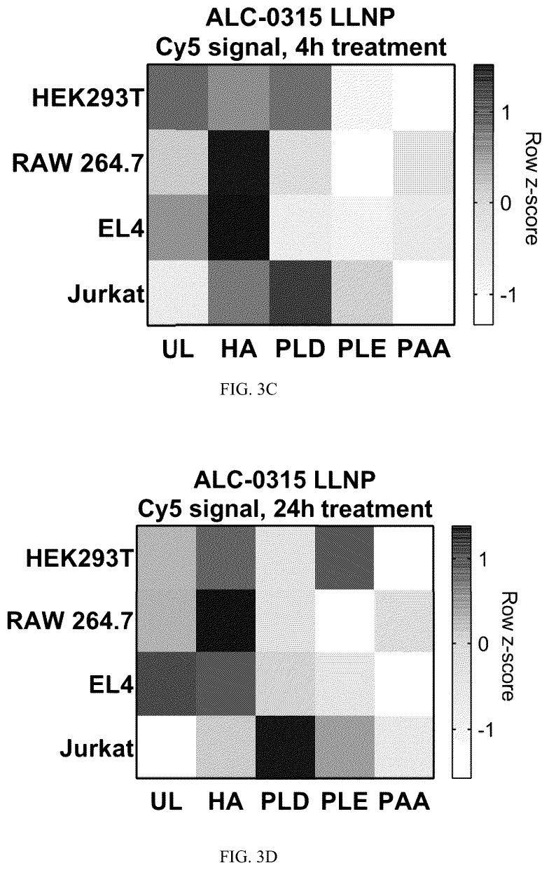

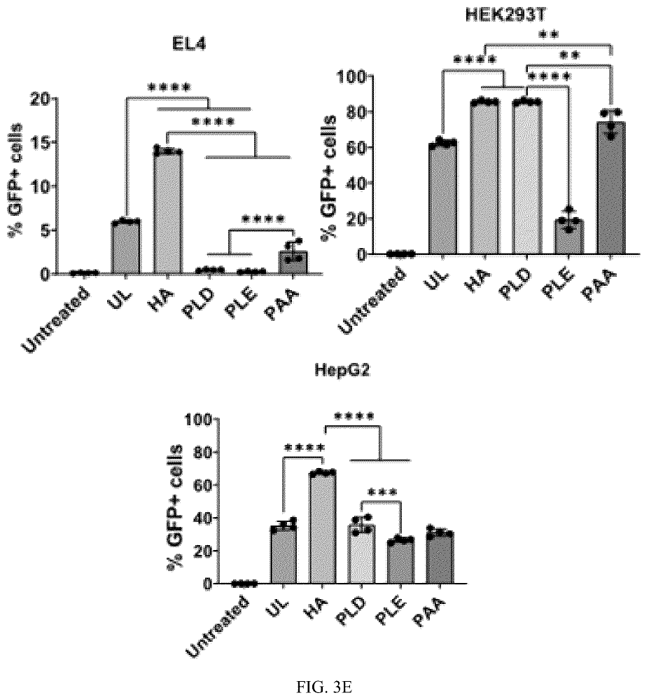

Comparisons of transfection after either 4 h or 24 h treatment suggested that polyanion coatings alter LNP uptake or RNA trafficking and release in a cell-specific manner (FIGS. 3A-B). Perhaps the most apparent trends are selective cell transfection based on the outer layer; for example, HA-LLNPs show significant uptake or association with each cell line examined in this study, but selective transfection in RAW 264.7 macrophages. PLE-LLNPs appear to drive much more transfection in HEK293T and Jurkat cells over EL4s and macrophages at longer timeframes. This behavior is particularly notable because macrophages tend to take up NPs at high rates compared to other cells; however, PLE-LLNPs do not show significant cell uptake or transfection in macrophages. After a 24 h treatment, PLD-LLNPs generated comparable or greater luciferase expression than unlayered LNPs, outperforming other layered formulations and providing strong transfection across these cell lines independent of cell association (FIG. 3B). On the other hand, PAA, which is a synthetic and highly charged polyanion, shows significantly lower uptake and relatively low transfection across each cell line. These observations contrast sharply with the unlayered LNPs, which exhibit entirely different trends in association and transfection. These results indicate that the polyanion layer coats the LNP surface and can meaningfully alter the trafficking, and ultimately the transfection, in different cell types.

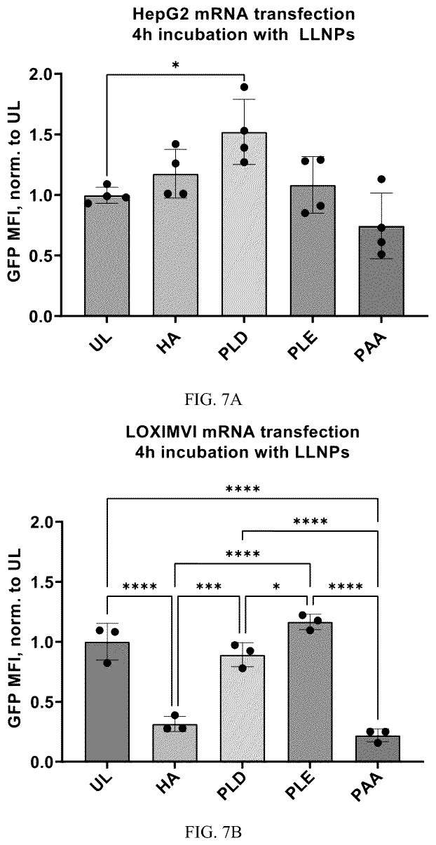

There are also meaningful differences in the apparent kinetics of association and transfection of the NPs. In HEK293 Ts, for example, PLE-LLNPs treated for 4 h induced significantly lower luciferase levels than did unlayered LNPs, which increased to much higher relative levels after 24 h treatment, suggesting this polyanion may direct trafficking in a manner that leads to slower mRNA delivery. Conversely, PAA-LLNPs after 4 h treatment generated comparable luciferase expression to unlayered LNPs; however, after 24 h treatment, PAA-LLNPs showed reduced luciferase levels relative to unlayered LNPs, suggesting that this layer may undergo trafficking that leads to either rapid uptake and clearance, degradation, or endosomal trapping within the first hours. mRNA delivery kinetics varied by cell type; in Jurkats, for example, PLD- and PLE-LLNPs treated for 4 h exhibited an increased luciferase expression over unlayered LNPs; these differences among groups decreased after 24 h treatment. In RAW 264.7s, HA-LLNPs treated for 4 h transfected comparably to unlayered LNPs, but transfection was greatly reduced relative to other groups at 24 h. Trends in LLNP mRNA transfection were also assessed in two cancer cell lines via flow cytometry, using LNP cores of the same lipid composition, containing EGFP-expressing mRNA (FIG. 7). LOXIMVI melanoma cells, dosed at 100 ng mRNA/well, exhibited similar trends in LLNP transfection; HepG2 liver carcinoma cells, dosed at 50 ng mRNA/well, exhibited less specificity for polyanion outer layers. Dead cells were excluded using a live/dead stain and MFI was determined via flow cytometry.

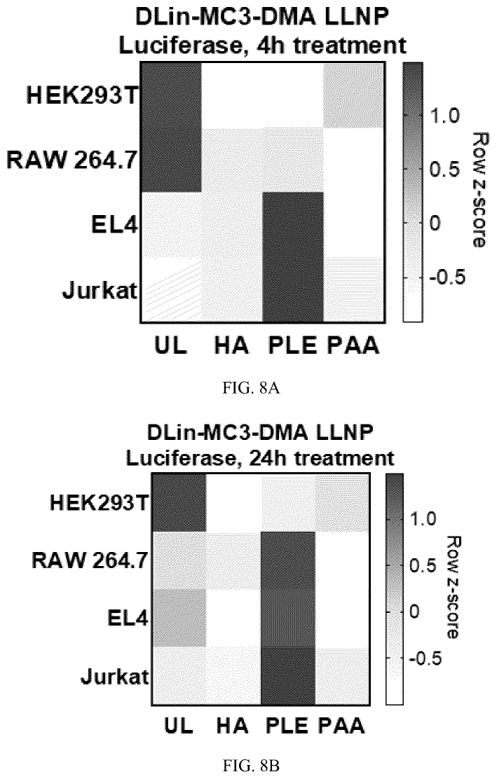

To assess the effect of layering on a different core, HA, PLE, and PAA were layered onto LNP formulations in a 50/38.5/1.5/10 ratio of DLin-MC3-DMA/cholesterol/DMG-PEG-2000/DSPC. Similar trends as those seen with the original cores were observed at 4 h and 24 h treatments across cell lines (FIG. 8). This data suggests that the LNP surface chemistry and resulting interactions are influenced by the polyanion outer layer coating, beyond the lipid composition.

To characterize LNP cellular association, cells were treated with LLNPs containing 0.5 mol % Cy5-labeled DOPE for either 4 h or 24 h; cellular Cy5 fluorescence was measured on a Tecan plate reader (FIGS. 3C-D). Notably, HA-LLNPs generated the highest Cy5 signal across cell lines at both incubations. PAA-LLNPs associated least across cell lines that similarly demonstrated reduced luciferase expression. Interestingly, though PLD-LLNPs generated the greatest luciferase expression, they associated moderately across cell lines. The discrepancy between cell association or uptake and transfection trends suggests that polyanions may further modulate the internal trafficking and RNA release.

To test the impact of polyanion adsorption on transfection of other nucleic acid cargos, ALC-0315 LLNPs were formulated containing GFP-encoding pDNA, layered with each of the four polyanions. pDNA delivery requires different intracellular release and trafficking profiles than mRNA delivery, since the former requires nuclear translocation for effective transfection. HEK293T, EL4, and HepG2 liver carcinoma cells were treated with LNPs for 4 h, then washed and cultured for an additional 20 h; GFP-positive cells were assessed via flow cytometry (FIG. 3E). Plasmid delivery across cell lines was enhanced most notably by HA-LLNPs, which more than doubled transfection rates. HA has previously improved lipoplex-mediated pDNA transfection, potentially due to intracellular interactions associated with its glycosaminoglycan structure (25). PLD- and PLE-LLNPs decreased pDNA-LLNP transfection in both EL4s and HEK293 Ts. Transfection rates in HepG2s, which divide and intake material rapidly, were not significantly impacted by PAA-, PLD-, or PLE-LLNPs.

Intracellularly, LLNP polyanions traffic with pDNA cargo. To examine the impact of the polyanion on intracellular trafficking of pDNA LLNPs, confocal microscopy was conducted on EL4s and HEK293 Ts treated with unlayered or layered LNPs for 4 h. These cell lines were chosen as representative adherent and suspension cultures. To facilitate co-tracking of lipid, nucleic acid, and polyanion, DOPE-Cy5 labeled LNPs were loaded with MFP488-labeled pDNA and layered with BDP 558/568-labeled polyanions.

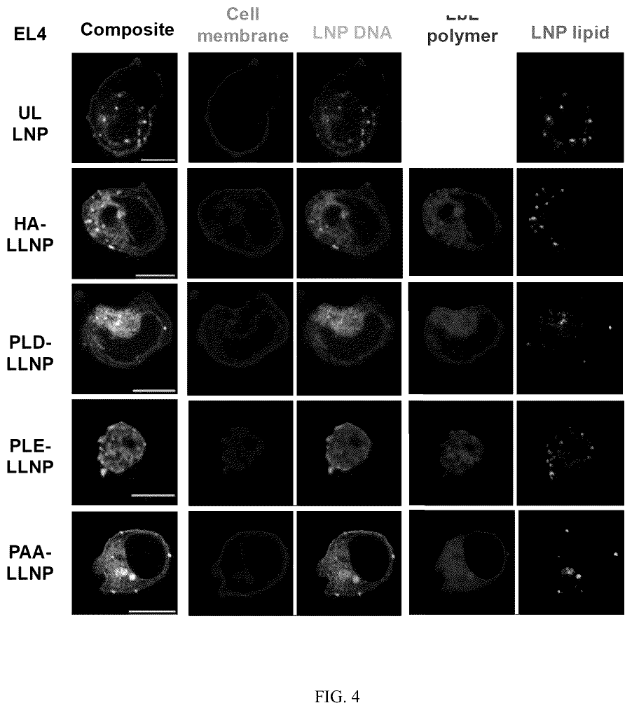

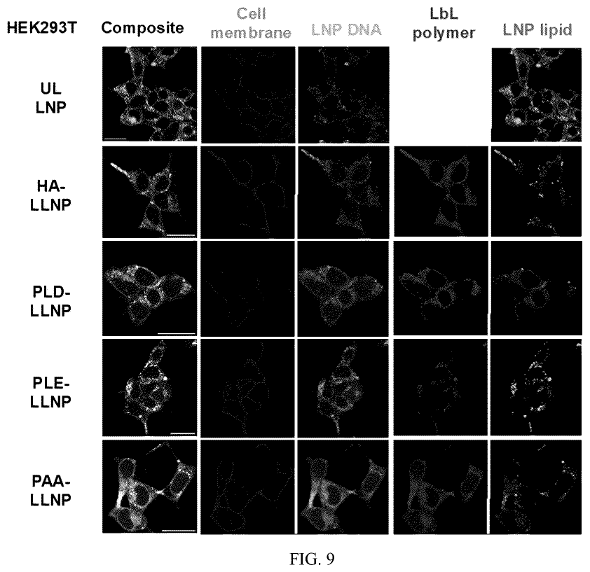

After 4 h treatment, EL4s internalized both unlayered and layered LNPs; polyanions were taken up along with core lipids and pDNA (FIG. 4). Across all groups, MFP488-pDNA signal appeared diffuse throughout the cytoplasm. EL4s treated with unlayered and HA-LLNPs additionally displayed some punctate pDNA signals co-localized with lipid signals; these NPs may be trapped in trafficking endosomes or lysosomes. pDNA nuclear localization was minimal, expected given the low rates of pDNA transfection in these cells. Though all groups exhibited some pDNA colocalization with the cell membrane, colocalization appeared strongest in cells treated with PLE- and PAA-LLNPs. In past studies, PLE has demonstrated localization to extracellular surfaces in cancer cell lines; similar cellular interactions may promote extracellular localization in EL4 lymphoma cells (22, 39). DOPE signal appeared punctate within the cytoplasm and cell membrane. Detected DOPE signal was significantly weaker than that of pDNA or polyanion; this may partially be due to the relative infrequency of labeled DOPE molecules in NP composition. Polyanion signal in LLNPs appeared diffuse in the cytoplasm, similar to pDNA dispersal. The relative positions of all three components suggest that polyanions and pDNA translocate through the cell in more similar patterns than do LNP lipids. HEK293 Ts exhibited similar internalization patterns; in addition, the three labeled LNP components were visualized trafficking between cells (FIG. 9).

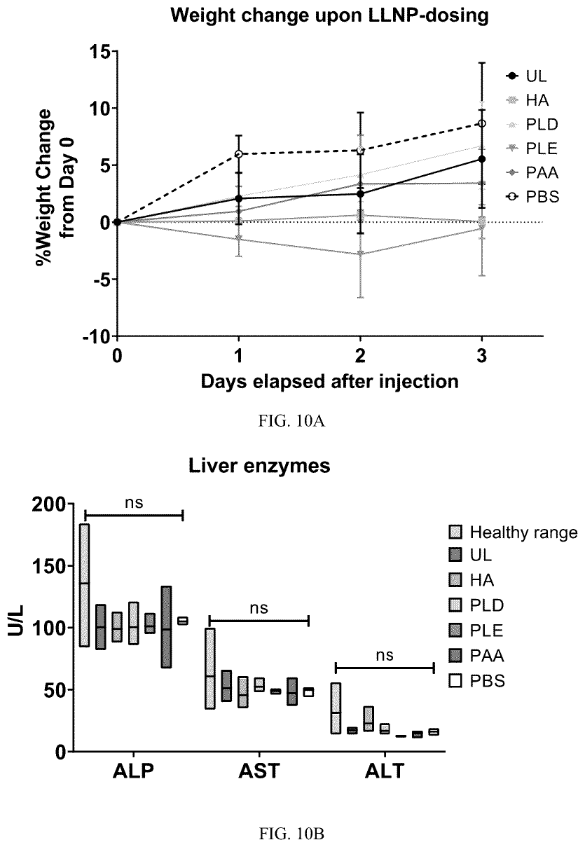

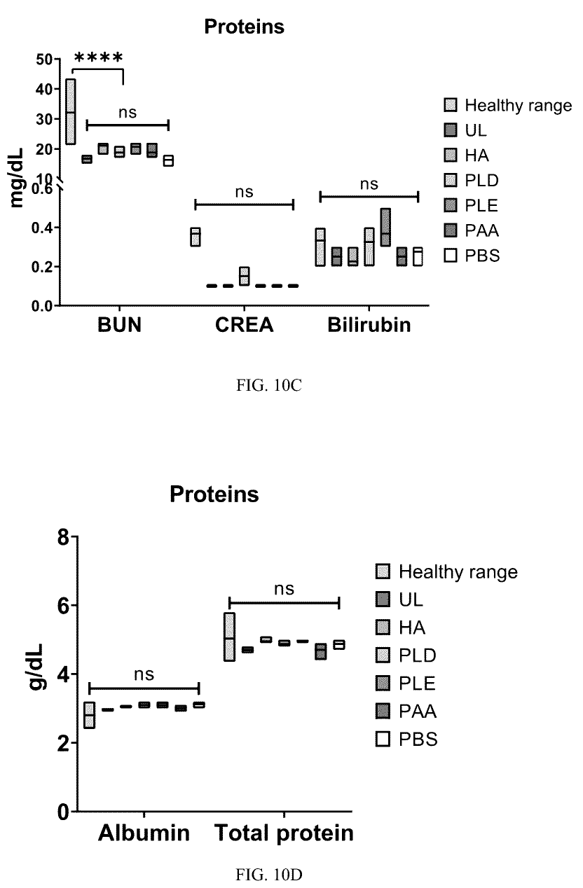

PLE and HA moderately improve hepatic and splenic transfection. Next, the impact of the surface polyanion on LNP biodistribution and transfection in healthy female C57BL/6 mice injected intravenously (i.v.) was impacted. LNPs with each of the four polyanions induced no toxicity 72 h after dosing, as measured by liver enzymes, serum protein, and weight changes (FIG. 10) (40, 41). Lower blood urea nitrogen (BUN) levels across groups, relative to literature-determined ranges, may be attributed to variability in vendor strains; however, no significant difference in BUN levels was observed between mice treated with LLNPs and mice treated with saline.

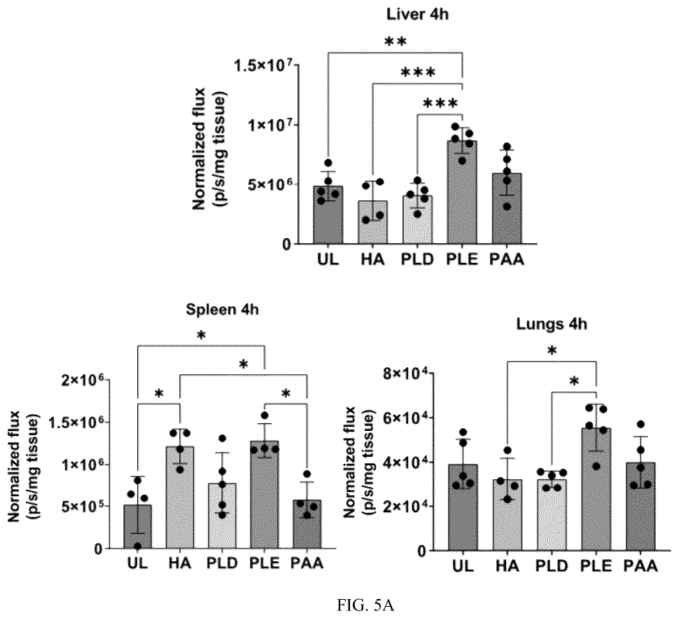

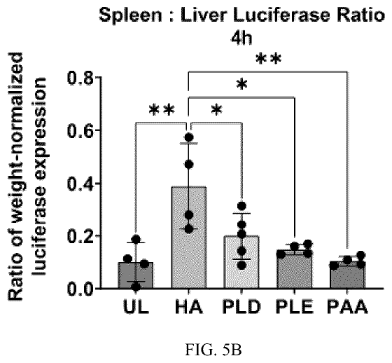

To study biodistribution and transfection, mice were dosed with unlayered and layered ALC-0315 LNPs at 0.3 mg/kg luciferase-encoding mRNA. Four hours after injection, the heart, lungs, liver, kidneys, and spleen were imaged for luciferase expression ex vivo. PLE-LLNPs increased liver and spleen luciferase expression 1.8-fold and 2.5-fold, respectively, over unlayered LNPs (FIG. 5A). HA-LLNPs increased spleen luciferase expression 2.3-fold over unlayered LNPs; however, this formulation did not increase liver transfection (FIG. 5A). In contrast to in vitro trends, PLD-LLNPs showed minimally improved or decreased transfection across organs; PAA-LLNPs performed similarly to unlayered LNPs in all organs studied. No significant differences from unlayered LNPs were detected in the heart or lungs for any polyanion. Comparing the ratio of weight-normalized flux in the spleen and liver, it was observed that HA-LLNPs showed higher preferential localization to the spleen than did other layers (FIG. 5B). In comparison, PLE-LLNPs increased expression in both liver and spleen.

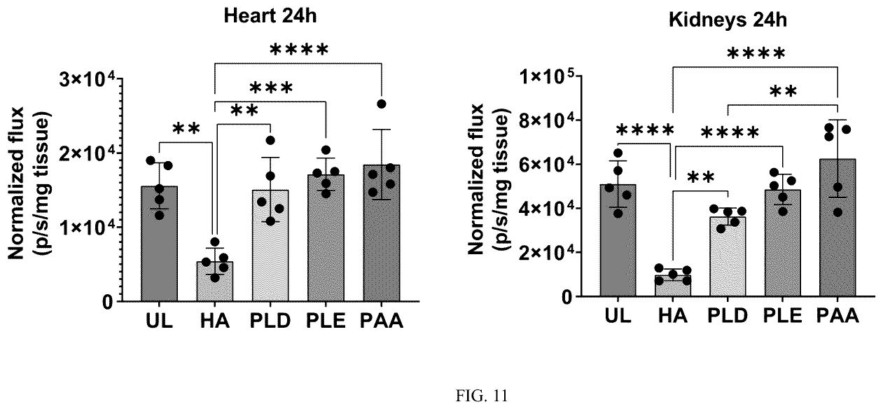

For a longer-term biodistribution study, organs were analyzed for luciferase expression 24 h after transfection. At 24 h, HA-LLNPs showed reduced luciferase expression across all measured organs (FIG. 5C). All other LLNPs performed similarly to unlayered LNPs, suggesting that the outer polyanion confers the greatest benefit at earlier times (FIGS. 5C, 11). Differences in transfection of LLNPs decrease over time, potentially due to stripping or degradation of the outer layer over time.

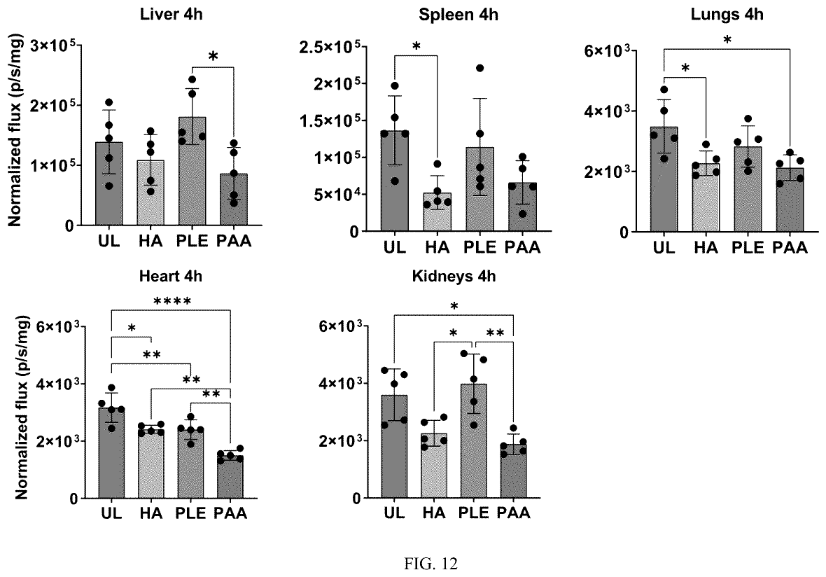

To compare trends in in vivo transfection across cores, 4 h organ-level transfection studies were conducted with LLNPs in which the core was composed of a 50/38.5/1.5/10 ratio of DLin-MC3-DMA/cholesterol/DMG-PEG-2000/DSPC (FIG. 12); PLD was excluded given minimal differences between PLD-LLNPs and unlayered LNPs in prior studies. Similar trends were observed by swapping the LLNP core; however most notably, HA-LLNPs with the alternate ionizable lipid decreased spleen luciferase expression 2.3-fold relative to unlayered LNPs (FIG. 12). The prominent difference in splenic performance may be attributed to synergistic impacts of both the polyanion and core helper lipids.

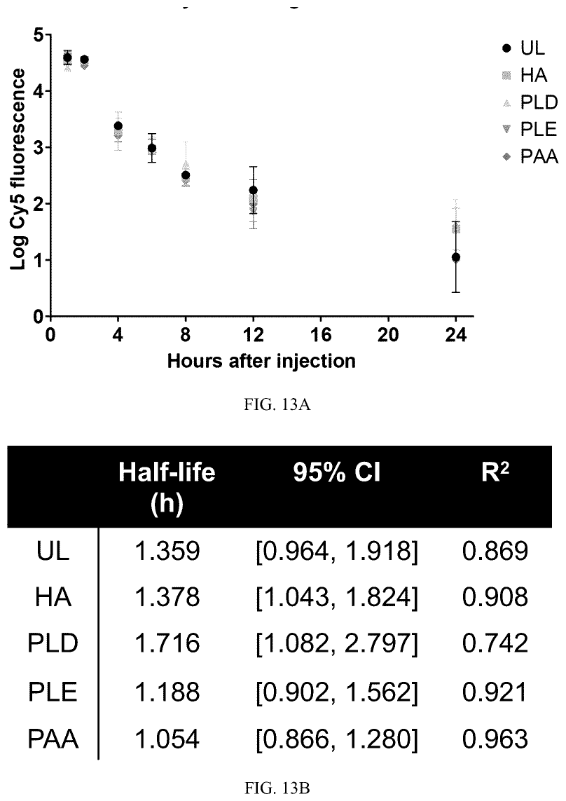

To evaluate the pharmacokinetics in vivo, mice were dosed i.v. with Cy5-labeled LNPs. Blood retention times were evaluated over a 24 h-period post-administration by quantifying the Cy5 signal in the blood serum (FIG. 13). Minimal differences in residence time were observed across LLNPs.

This example demonstrates the inventors' provision of a method to stably deposit charged bioactive polymers onto LNP surfaces, forming LLNPs, as a tunable modality to alter the NP's surface presentation and cellular interactions both in vitro and in vivo. Polyanion chemistries can modulate selectivity in LNP uptake and transfection, helping to address the ongoing challenge of targeted nucleic acid delivery.

The LbL assembly enables the incorporation of a wide variety of charged polymers in a modular fashion; many types of polymers can be stably layered onto a diverse range of LNP cores varying in component lipids and nucleic acid cargos. Relative to lipid-centered LNP development, which requires the synthesis of novel compounds and iterative optimization of LNP formulations (15, 16), the electrostatic adsorption of surface polymers can be easily applied to already-optimized LNP cores. Indeed, an optimized LNP delivery vector may benefit significantly from the combination of a highly efficacious ionizable lipid to boost transfection and a rationally chosen surface polyanion to provide on-target selectivity.

Polyanion chemistry impacts LNP apparent pKa and colloidal stability, while maintaining LNP structure and encapsulation of nucleic acid cargos (FIGS. 1A-E, 2A-C). Furthermore, a stably adsorbed layer of polyanion helps ensure colloidal stability without incorporating additional PEG and PEGylated compounds; the polyanion may also shield LNP PEG-chains from generating or reacting with human PEG antibodies (11). While recent works have conjugated active targeting elements, such as antibodies, peptides, or even polymers, to LNP surfaces (42-46), the LbL assembly allows the non-covalent attachment of polyanions capable of inherent targeting and specificity. In addition, LbL assembly enables the facile incorporation of diverse chemistries onto LNP surfaces. Rational choice of polyanions, which include many known biomolecules such as polysaccharides and polypeptides, can leverage cell-specific receptor expression to improve the specificity of NP association. In conjunction with this capability to choose a layer with specific cell interactions, the strongly negative charge and hydrated brushy nature of the polyanion outer layers on LLNPs reduce nonspecific protein adsorption that can lead to undesired uptake and transfection of off-target cells and tissues (FIG. 2C).

For example, HA is a natural ligand for CD44, a receptor overexpressed by endothelial cells, macrophages, and certain cancer cells (31, 47). LLNPs with adsorbed HA showed higher luciferase signal and Cy5 uptake in RAW 264.7 macrophages, known to express CD44, than other LLNPs (FIGS. 3A, C) (47, 48). Moreover, at 4 h treatment, other cell lines showed significantly reduced transfection by HA-LLNPs. This selectively increased uptake can enable delivery specific to cell phenotypes expressing natural binding sites for HA; for example, HA-conjugated LNPs have been used for targeting of ovarian and glioblastoma multiforme (GBM) cancer cells (43, 44). Both HA and PLD have also been adsorbed onto liposomal NPs to improve uptake by ovarian cancer cells in murine models (22, 23, 39). Given that these polyanions have multiple disease-relevant biological binding partners, LbL assembly may enable future LNP targeting strategies for cancer or immunotherapy applications without the need for bioconjugation of additional ligands. By contrast, the relatively low uptake and transfection that was observed with PAA-LLNPs may arise because PAA is a bioinert polymer that may not benefit from receptor-mediated uptake mechanisms. These properties may make PAA a desirable “stealth” layer to minimize off-target uptake, for cases in which an additional ligand is utilized.

Prior studies have demonstrated that LNPs with acidified apparent pKa negative surface charge preferentially localize to the spleen over the liver; the opposite trends induce preferential localization to the lungs (18, 19, 38). In this study, PLE-, PLD-, and PAA-LLNPs all and exhibited acidified pKa and negative charges of similar magnitudes; notably, only PLE-LLNPs improved splenic transfection. Variability in localization and transfection may be attributed to affinities between the outer polyanion and receptors of cells of particular phenotypes. PLE- and HA-LLNPs showed improved LLNP luciferase signal 4 h after injection but did not perform similarly 24 h after injection. Cellular subtype profiling may indicate whether drops in luciferase signal are due to the metabolic rate of the cell subtypes primarily transfected by these NPs. Different polymer coatings have been shown to uniquely modify the protein corona of polymeric NPs, which may impact uptake and cell phenotype selectivity (49). Furthermore, prior work has demonstrated that the polyanion identity impacts intracellular trafficking and lysosomal clearance pathways (22).

The use of electrostatics to decorate NP surfaces is a powerful tool that can be generalized to facilitate incorporation of other therapeutic or targeting moieties into LNP delivery systems.

The LNP layering method described herein can be adapted to incorporate additional targeting and therapeutic modalities into these highly potent delivery vehicles, to improve on-target and selective uptake and transfection, deliver combination therapies, and harness receptor-mediated interactions to boost transfection of difficult cell types.

Materials and Methods. Dilinoleylmethyl-4-dimethylaminobutyrate (DLin-MC3-DMA), N,N-dimethyl-2,2-di-(9Z,12Z)-9,12-octadecadien-1-yl-1,3-dioxolane-4-ethanamine (DLin-KC2-DMA), and 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200) were purchased from MedChemExpress. 1,2-dimyristoyl-rac-glycero-3-methoxypolyethylene glycol-2000 (DMG-PEG-2000) and 3,6-bis[4-[bis(2-hydroxydodecyl)amino]butyl]-2,5-piperazinedione (cKK-E12) was purchased from Cayman Chemical Company. Cholesterol, 1,2-distearoyl-sn-glycero-3-phosphocholine (DSPC), 1,2-dioleoyl-sn-glycero-3-phosphocholine (18:1 (A9-Cis) PC), 1,2-dioleyol-sn-glycero-3-phosphoethanolamine (18:1 (A9-Cis) PE), 1,2-dioleyol-sn-glycero-3-phosphoethanolamine-N-(Cyanine-5) (18:1 Cy5 PE), and 6-((2-hexyldecanoyl)oxy)-N-(6-((2-hexyldecanoyl)oxy)hexyl)-N-(4-hydroxybutyl)hexan-1-aminium (ALC-0315) were purchased from Avanti Polar Lipids.

CleanCap Firefly Luciferase mRNA (5-methoxyuridine) was purchased from TriLink Biotechnologies. The Label IT Nucleic Acid Labeling Kit (MFP488) was purchased from Mirus Bio. The Quant-it RiboGreen RNA Assay Kit and Quant-it PicoGreen dsDNA Assay Kit were purchased from Fisher Scientific.

Sodium hyaluronate was purchased from Lifecore Technologies. Polyacrylic acid, sodium salt (PAA, 15 kDa) solution, N-hydroxysuccinimide ester (NHS), and N-(3-dimethylaminopropyl)-N-ethylcarbodiimide hydrochloride (EDC) were purchased from Sigma Aldrich. Poly-L-glutamic acid sodium salt (PLE100, 15 kDa) and poly-L-aspartic acid, sodium salt (PLD100, 14 kDa) were purchased from Alamanda Polymers. MWCO dialysis membranes were purchased from Repligen. BDP-558/568-amine was purchased from Lumiprobe.

Cell culture. Cell lines used for this study included: HEK293 Ts, EL4s, RAW 264.7, HepG2, LOXIMVI, Jurkat. HEK293T, EL4, and RAW 264.7 cells were purchased from ATCC. LOXIMVI and Jurkat cells were a gift from the Straehla Lab. HepG2 cells were sourced from the Koch Institute's High Throughout Sciences Facility. HEK293T, EL4, and RAW 264.7s were cultured in Dulbecco's Modified Eagle Medium (DMEM) supplemented with 10% fetal bovine serum (FBS) and 1% penicillin-streptomycin (Pen-Strep). HepG2s were cultured in Eagle's Minimum Essential Medium (EMEM) supplemented with 10% FBS. Jurkats and LOXIMVIs were cultured in Roswell Park Memorial Institute (RPMI) media supplemented with 10% FBS, 1% Pen-Strep. All cells were cultured in a humidified chamber at 37° C. and 5% CO₂, and handled in sterile conditions. Cell cultures were tested for mycoplasma contamination using a MycoAlert kit (Lonza), in the High Throughput Sciences core and the ES Cell and Transgenics Facility at MIT's Koch Institute for Integrative Cancer Research. All cell lines were used up to passage 20.

LNP formation. DSPC, DOPC, DOPE, and DOPE-Cy5 were dried from chloroform stocks under nitrogen, then dissolved in 100% ethanol. Cholesterol, DLin-MC3-DMA, DLin-KC2-DMA, and DMG-PEG-2000 were directly dissolved in 100% ethanol. LNP lipid stocks were formed at varying molar compositions (FIG. 14).

Nucleic acid cargo (either mRNA or pDNA) was dissolved in 25 mM sodium acetate in a 4-mL scintillation vial. To 4 volumes of nucleic acid under stirring, 1 volume of lipid mixture in 100% ethanol was pipetted in rapidly. The solution was rested without stirring for 5 min. Then, under resumed stirring, the solution was diluted with 5 volumes of DNAse/RNAse free water. Ethanol and sodium acetate were removed via dialysis against either water or 1×PBS for 4 h. LNPs were concentrated using Amicon Ultra-4 ultracentrifugal filter units, MWCO 100K. LNP solutions were stored at 4° C. and used within 3 days of preparation.

LNP characterization. LNP hydrodynamic diameter, polydispersity index, and zeta-potential were measured on a Malvern Zetasizer Pro (Malvern Panalytical) with a red laser and a detection angle of 173°.

LNP encapsulation efficiency. Encapsulation efficiency of mRNA and pDNA was determined with the Quant-it RiboGreen RNA assay kit and PicoGreen dsDNA assay kit, respectively. In a Nunc F96 MicroWell Black polystyrene plate, 5 μL of LNP samples were incubated in 45 μL of either 1×TE or 0.5% (v/v) Triton X-100 solution in 1×TE. Samples were shaken at 130 rpm, at 37° C., for 10 minutes. RiboGreen reagent was diluted 200-fold in 1×TE and protected from light. Samples were then mixed with 50 μL of the diluted RiboGreen reagent. Then, samples were shaken at 300 rpm at room temperature (RT) for 5 min, protected from light. Fluorescence intensities were read immediately on a Tecan M1000 plate reader, at an excitation of 485 nm and emission of 525 nm. Encapsulation was calculated as: (Fluorescence of Triton X-100 LNPs−Fluorescence of TE LNPs)/(Fluorescence of Triton X-100 LNPs).

LNP layering. Polyanions were dissolved in nuclease-free water to concentrations of 10-20 mg/mL, then sonicated for 10 min. Layering baths were prepared by diluting polyanion stocks into 5 mM HEPES buffer (starting stock at pH 7.2). Stocks of HA, PLD, PLE, and PAA were diluted to 8, 2, 2, and 1.2 respective weight equivalents, relative to LNP lipid concentration. The polyanion bath was added to a 4-ml scintillation vial and stirred at 800 rpm at RT, to which an equal volume of LNPs in water was added. The mixture was stirred for 15 min, then incubated at RT for 1 h. Layered LNPs were then purified and concentrated via 3 water washes in Amicon Ultra-4 ultracentrifugal filter units, MWCO 100K. Layered LNPs were stored at 4° C.

Dye-labeled polymer synthesis. Carboxylated polymers were fluorescently labeled with BDP 558/568-amine by NHS/EDC coupling chemistry. Polymers (HA, PLD, PLE or PAA) were diluted to 2 mg/mL in MES buffer (0.1 N, pH 6). Then, solutions of BDP 558/568-amine in DMSO, and of NHS and EDC in MES buffer, were added at a concentration 6.3-(0.63- for PLE), 14.3-, and 143-fold times higher than the polymer, respectively. The solution was stirred for 4 h at RT, then dialyzed against NaCl 50 mM overnight and against ultra-pure water for up to 72 h to remove free reagents. A 3.5 KDa regenerated cellulose membrane was used for dialysis (Spectrum Labs). The final polymer solution was frozen at −80° C. and lyophilized in a Labconco FreeZone Freeze Dryer System.

Characterization of plasma proteins adsorbed to LLNPs. Adsorbed serum proteins on LLNPs were characterized using a previously described method (38). Unlayered or layered mRNA-LNPs were diluted to 0.4 mg/mL lipid. characterize adsorbed serum proteins, unlayered or layered mRNA-LNPs containing DOPE-Cy5 were incubated in an equal volume of 100% mouse plasma for 1 h at 37° C. The solution was then centrifuged at 25,000 rcf at 4° C. for 2 h. The supernatant was removed, and the pellet was washed with nuclease-free water. The pellet was washed twice in water via centrifugation at 25,000 rcf at 4° C. for 2 h, then resuspended in 50 μL water. A volume of 5 μL of each pellet was diluted into 95 μL DMSO in a black 96-well flat-bottom plate, and Cy5 fluorescence was read on a Tecan M1000 plate reader (Ex/Em 630/670 nm). Pellets were diluted to equal Cy5 concentrations. The concentration of protein in each pellet was determined using the Pierce BCA Protein Assay Kit; standard curves for the BCA assay were prepared using Cy5-mRNA LNPs as background.

Transfection studies. Adherent cells were seeded in 96-well flat-bottom plates at 10,000 cells/well in 90 μL of supplemented cell media and allowed to grow at 37° C., 5% CO₂, for 24 h prior to treatment with LNPs. Suspension cells were seeded at 25,000 cells/well in 90 μL of complete cell media, and were treated immediately with LNPs. For 24 incubations, cells were not washed. For 4 h incubations, cells were washed and re-suspended in fresh media 4 h after transfection, then incubated for an additional 20 h before readout.

Luciferase and cell viability assay. Adherent cells were washed in 100 μL 1×PBS, treated with 30 μL 0.25% Trypsin-EDTA for 5 min, diluted with 150 μL complete media, washed, and resuspended in complete media. Suspension cells were washed in 100 μL 1×PBS, then resuspended in complete media.

Half of the cell volume was plated in a black 96-well flat-bottom plate and diluted to 100 μL per well with complete media. A volume of 10 μL of PrestoBlue viability reagent were added to each well. Samples were incubated for 30 min at 37° C., 5% CO₂, protected from light. PrestoBlue fluorescence was read at an excitation of 560 nm and emission of 600 nm on a Tecan M1000 plate reader. The remaining half of the cell volume was plated in a clear 96-well plate, washed in 1×PBS, then lysed with 1×Lysis Buffer for 15 min at room temperature while shaking. 20 μL of cell lysate were plated into a white flat-bottom 96-well plate, and mixed with 100 μL of 0.2 mg/mL D-luciferin, diluted in Biotium Firefly Luciferase Assay Buffer 2.0. Immediately after adding D-luciferin, luminescence was read on a Tecan M1000 plate reader, using an integration time of 1000 ms. Background signal from untreated cells was subtracted from all luminescence values.

Animal experiments. All animal experiments were approved by the MIT Committee on Animal Care (protocol #221000434).

Statistical analysis. Statistics were calculated using GraphPad Prism. Multiple comparisons among groups were analyzed using a one-way ANOVA test, with Tukey's multiple comparisons post hoc test. Differences were considered statistically significant if the calculated p-value was less than 0.05. All data are presented as a mean value with standard deviation (mean±SD).

### Example 2: Impact of Surface Chemistry on Lipid Nanoparticle Transfection & Targeting of Hematopoietic Stem & Progenitor Cells

Lipid nanoparticles (LNPs) are potent transfection agents for multiple nucleic acid cargos, including mRNA and plasmid DNA. LNPs can therefore mediate in vivo therapeutic delivery to the bone marrow, which expresses genetic diseases such as sickle cell disease and hemophilia. However, intravenous LNP delivery to extrahepatic spaces is challenged by multiple physiological parameters. NP surfaces adsorb negatively charged serum proteins, which induce aggregation and activate rapid sequestration by immune cells (53). In particular, LNPs adsorb apolipoprotein E (ApoE), which enhances accumulation in the liver by binding to hepatocytic low-density lipoprotein (LDL) receptors (10). Moreover, efficient gene delivery must mitigate off-target LNP uptake by more populous cell types. LNP surfaces for targeted extrahepatic gene delivery can therefore benefit from surface modifications.

The present disclosure provides LNPs layered with surface polyanions using layer-by-layer electrostatic deposition that not only minimize off-target uptake, but also facilitate covalent attachment of moieties to improve endocytosis and intracellular trafficking and have advantageous nanoparticle biodistribution when delivered intravenously, which is advantageous for extrahepatic delivery.

This example relates to an application of the LLNP technology to develop nanosystems capable of targeting and transfecting hematopoietic stem and progenitor cells (HSPCs) in vivo and ex vivo, with nucleic acid payloads including but not limited to mRNA, pDNA, Cas9 mRNA+sgRNA, ribonucleoprotein (RNP) complexes, or siRNA.

HSPCs are a high-potential therapeutic target. However, non-viral transfection of these cells is challenged by the facts that (1) native HSPCs reside in a quiescent state outside the cell cycle, and are therefore resistant to particle uptake and transfection, and (2) in vivo therapies are more likely to be taken up by other, more populous hematopoietic populations in the peripheral blood, including myeloid and lymphoid cells. The targeted LLNPs disclosed herein address these two challenges.

Antibody modification. Antibodies were purchased commercially available sources, and purified of sodium azide if necessary. Antibodies were reduced with 32 or more molar equivalents of TCEP in 20 mM phosphate, 150 mM NaCl, 10 mM EDTA (pH 7.2) for 1 h at 37° C. Sulfo-maleimide-PEG4-DBCO linkers were then conjugated to antibodies at room temperature, shaking at 300 rpm, for 1 h, protected from light. Antibodies were purified by passing through Zeba spin columns, primed with four volumes of PBS.

Ab-LLNP conjugation. Azide-modified PAA was prepared by stirring PAA, azidopropylamine, N-hydroxysuccinimide, and ethyl-3-(3-dimethylaminopropyl) carbodiimide for 4 h at RT. The polymer was then dialyzed against 50 mM NaCl for 24 h, then against water for 3-4 days, changing dialysis solution twice a day. Polymers were then lyophilized and characterized via FTIR and 1H-NMR. Average polymer modification of approximately 4%.

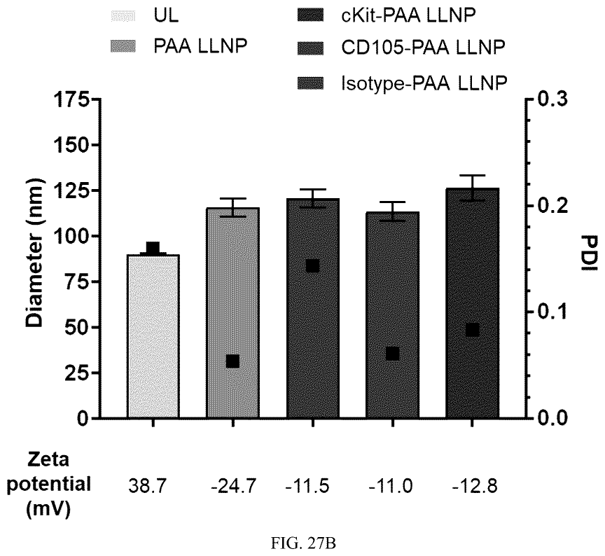

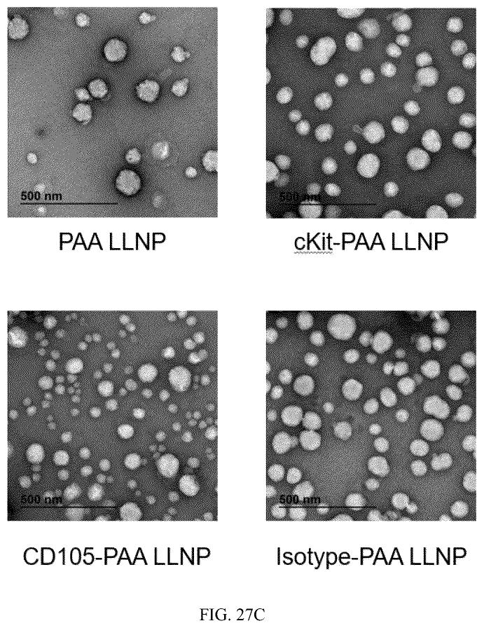

To prepare Ab-LLNPs (FIG. 27A), LNPs were first layered with a mixture of 90% PAA (15 kDa) and 10% azide-modified PAA. LLNPs were resuspended in 1×PBS and mixed with three weight equivalents of DBCO-conjugated antibody, shaking at 300 rpm, RT, for 1 h, then 4° C. for 18-24 h. Ab-LLNPs were then dialyzed against 1×PBS for 24 h in 300 KDa Float-a-Lyzers, to remove unreacted antibodies. Antibodies used for mouse HSC targeting include: cKit/anti-CD117 (clone 2B8, IgG2b K), anti-CD105 (clone MJ7/18, IgG2b κ), and a non-targeting isotype.

HM-1 studies. Female B6C3F1 mice, 6-8 weeks of age, were inoculated with 1 M OV2944-HM-1 cells. 10 days after inoculation, mice were dosed intraperitoneally with unlayered or LLNPs containing mCherry-encoding mRNA and a lipid-fluorophore DiD. Both 4 and 24 h after dosing, mice were sacrificed and the following organs were collected from the peritoneal space: liver, kidneys, spleen, tumored omentum, tumored urogenital tract (UGT). Organs were placed in Roswell Park Memorial Institute (RPMI) media on ice during harvesting and protected from light, then weighed and transferred to PBS. Organs were then imaged ex vivo on IVIS.

Genomic DNA extraction. LNP-treated cells were washed twice in 100 uL PBS, then resuspended in 200 μL of 10 mM Tris-HCl, 0.05% sodium dodecyl sulfate (SDS), and 100 μg/ml proteinase K. Cells were incubated at 37° C. for 1.5 h, shaking at 130 rpm. Cell lysates were heat inactivated at 80° C. for 30 min. Resulting concentrations of genomic DNA were measured with NanoDrop. PCR reactions were prepared, mixing genomic DNA, primers for target gene Rosa26, Taq master mix (NEB), and nuclease-free water. PCR was conducted on a Thermal Cycler with the following program: 95° C. 30 s, 30 cycles of (95° C. 15 s, 53° C. 30 s, 68° C. 22 s), then 68° C. for 5 min. PCR products were immediately put on ice.

PCR products were loaded onto 1% agarose gels, and run for 10 min at 160 V, 13 W. Target amplicons were excised from gels and purified using Qiagen QIAquick Gel Extraction Kit. Amplicons and primers for Rosa26 were submitted for Sanger sequencing by Quintara Biosciences. Indels were calculated using the online TIDE tool.

In vivo SCD targeting & bone marrow harvest. Ab-LLNPs were dosed intravenously into Ai14 mice via retro-orbital injection. After 72 h, approximately 0.5 ml blood was collected from each mouse via retro-orbital bleeds prior to sacrificing. Liver, spleen, lungs, kidneys, heart, femurs, and tibia were collected from each mouse and stored in RPMI on ice, protected from light. After organs were weighed, they were transferred to PBS and imaged on IVIS. Bone marrow cells were extracted from 1 femur and tibia per mouse. Bone ends were cut, then bones were spun through 70 um cell strainers into Eppendorf tubes at 1000×g for 20 min. To lyse red blood cells, cell pellets were incubated in ACK lysing buffer for 3-5 min, spun at 300×g for 5 min, and washed twice. Cell concentrations of remaining white blood cells were measured. Cells were stained for: Sca-1 (Pacific Blue), viability (BV510), cKit ACK2 (FITC), CD150 (PE-Cy7), CD48 (APC), and lineage (AF700).

Peripheral blood was collected in heparinized tubes, then spun at 300×g for 20 min. To lyse red blood cells, cell pellets were incubated in ACK lysing buffer for 3-5 min, spun at 300×g for 5 min, and washed until no red blood cells were visible. Cell concentrations of remaining white blood cells were measured. Cells were stained for: B220 (Pacific Blue), viability (BV510), CD45 (FITC), CD11b (PE-Cy7), CD3e (APC), and Ly6/G (BV605).

To prepare cells for flow cytometry, cells were washed 1× in PBS, incubated in 100 uL Zombie Aqua diluted 500× in PBS for 10 min at RT protected from light, and neutralized in Cell Staining Buffer. Cells were then incubated in 100 uL Fc-block in Cell Staining Buffer for 10 min at 4° C., washed, then incubated in 100 uL antibody stains for 25 min at 4° C. Cells were washed 2× in Cell Staining Buffer, then run through flow cytometry on a BD Fortessa HTS-2 analyzer.

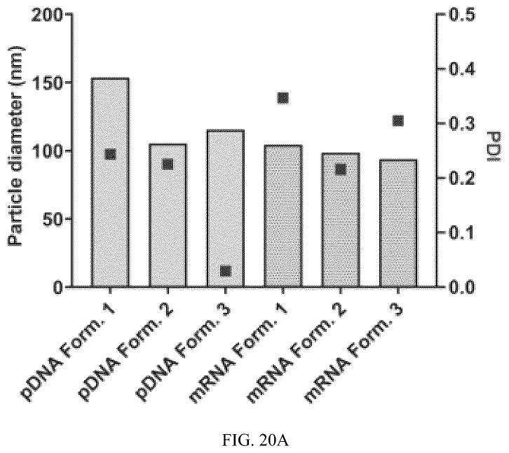

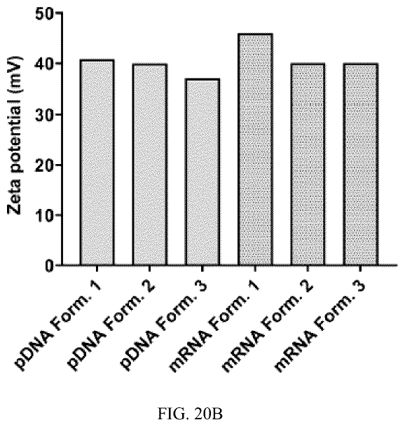

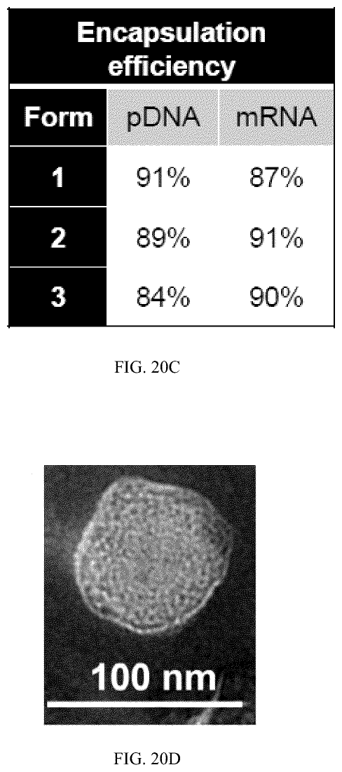

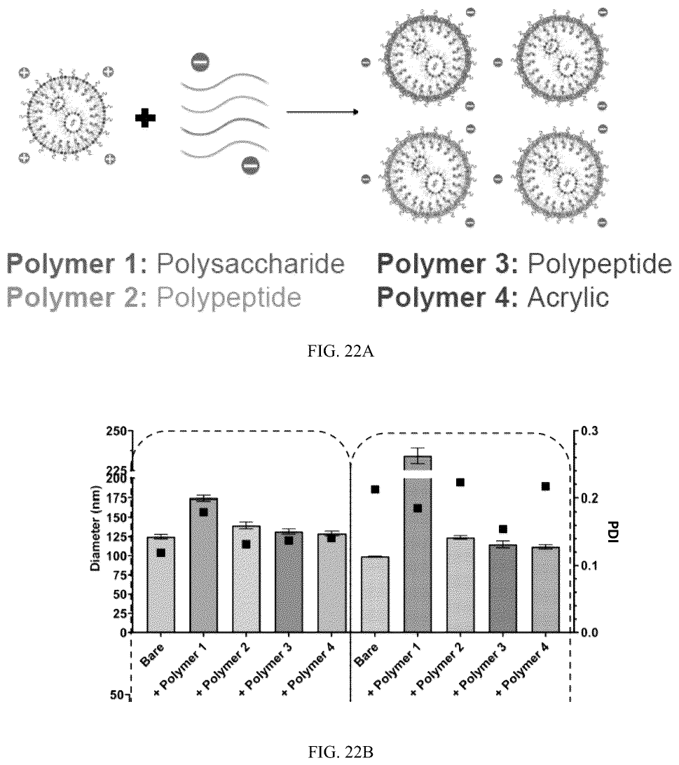

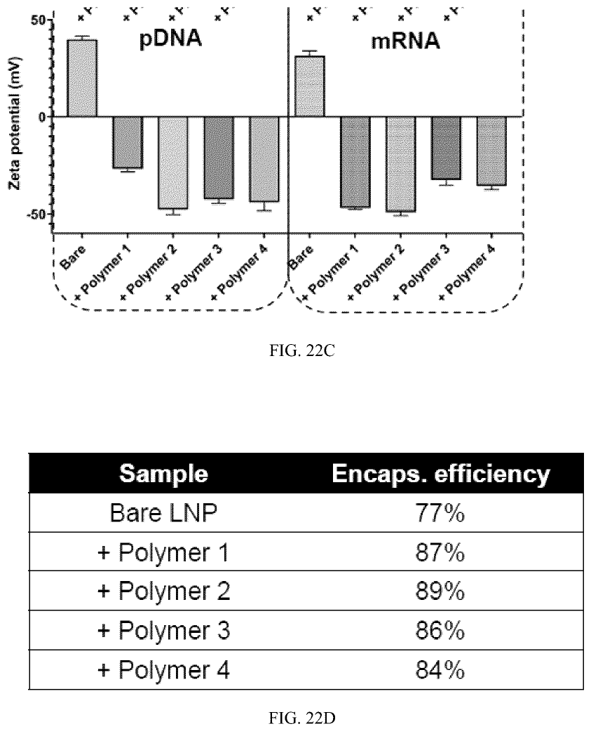

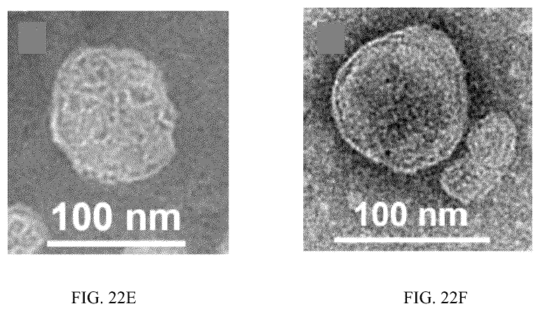

Results and Discussion. To achieve significant mRNA transfection of HSPCs, LNP core lipid identities and molar formulations were optimized and characterized (FIGS. 20-23). Certain optimized LNP cores according to the present disclosure include: (1) ionizable cationic lipid cKK-E12, C12-200, ALC-0315, SM-102, DLin-MC3-DMA, or DLin-KC2-DMA (20-50 mol %), (2) phospholipid DOPE (4-20 mol %), (3) PEG-lipid DMG-PEG-2000 (0.5 to 3.5 mol %), and (4) cholesterol (30 to 75 mol %). LNPs range from 60-200 nm in diameter and achieve nucleic acid encapsulation efficiencies of 40-95% (FIGS. 20C, 22D). It was determined that, relative to unlayered LNPs, layered LNPs experienced diameter increases of 5 to 50 nm (FIG. 22B) and furthermore, that upon layering, cationic LNPs experienced complete charge reversal; layered LNPs exhibited surface potentials of −27 to −48 mV (FIG. 22C). Electron micrographs (FIGS. 22E-F) show relative to unlayered mRNA LNPs (FIG. 22E), layered LNPs (FIG. 22F), which exhibited similar size increases and charge reversals, retain internal core structure. The formed LNPs can be purified by dialysis or ultracentrifugation.

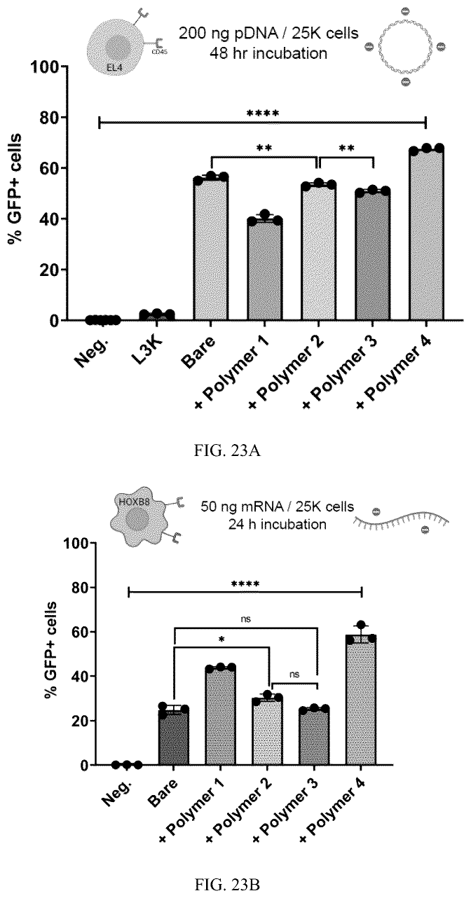

In EL4 cells, layered pDNA LNPs achieved comparable or higher transfection than unlayered pDNA LNPs and LNPs layered with Polymer 4 achieved 68% transfection, relative to bare 56% (FIG. 23A). In ER-HOXB8 cells, layered mRNA LNPs demonstrated improved transfection over unlayered mRNA LNPs and LNPs layered with Polymer 4 achieved 59% transfection, relative to bare 25% (FIG. 23B).

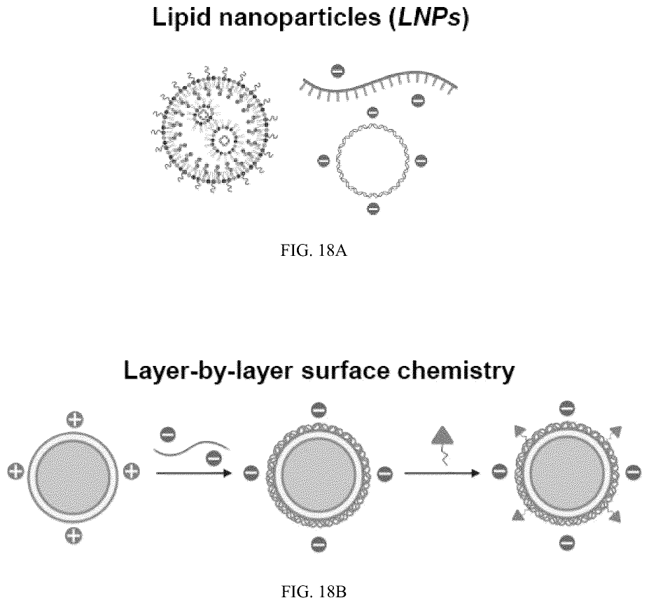

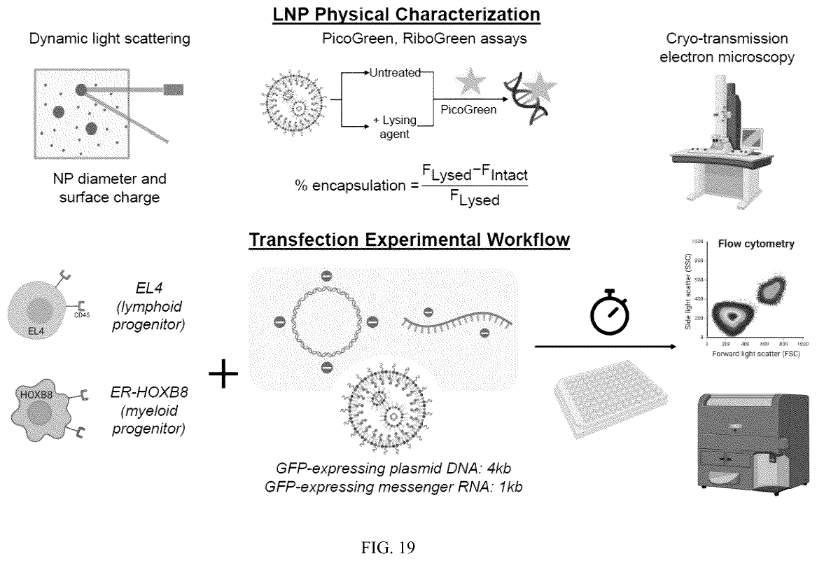

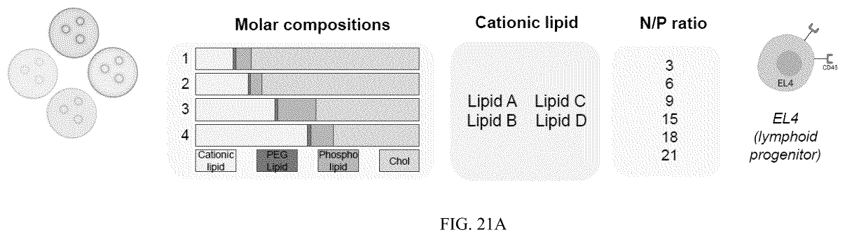

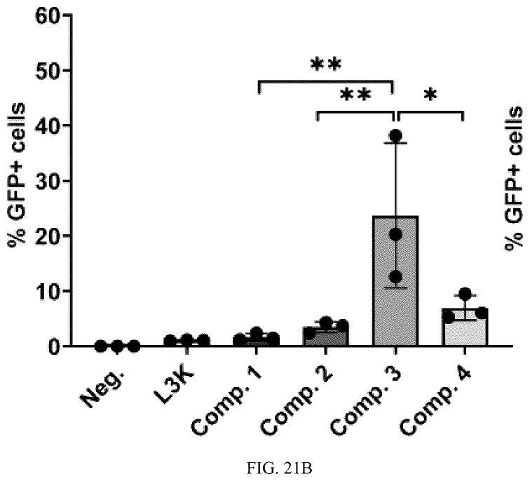

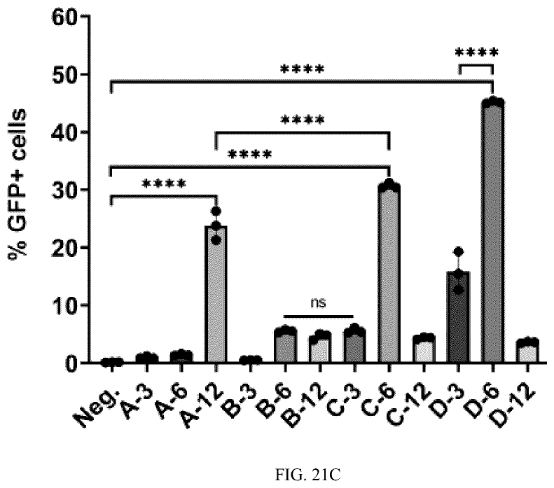

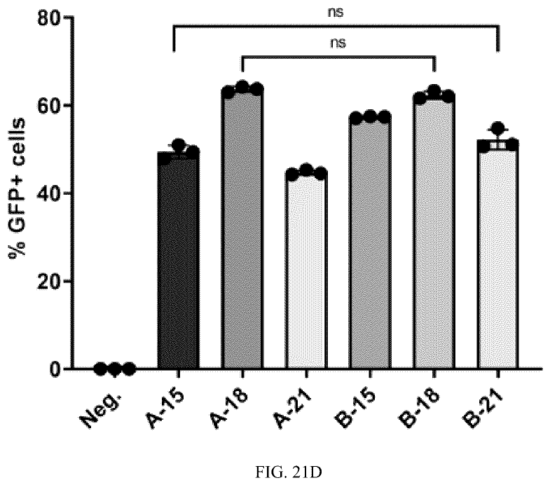

GFP-LLNPs. GFP-expressing mRNA or plasmid DNA was encapsulated in LNPs, then carboxylated surface polyanions containing either polypeptides or acrylic groups were deposited (FIG. 18A). Transfection potential was assayed in vitro in EL4 lymphoma cells and ER-HOXB8 progenitor cells via flow cytometry (FIGS. 19, 23A-B). It was determined that molar composition 3 induced optimal transfection (FIG. 21B). Furthermore, using the molar composition 3, a library of pDNA LNPs varying cationic lipids and N/P ratios was screened in EL4s (FIGS. 21C-D). The highest-performing combinations, A18 and B18, achieved >60% transfection after 48 h incubation (FIG. 21D).

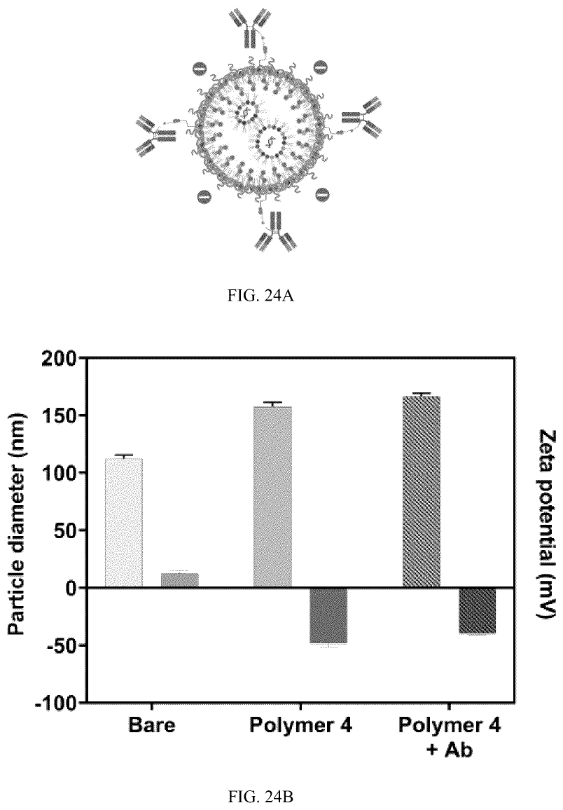

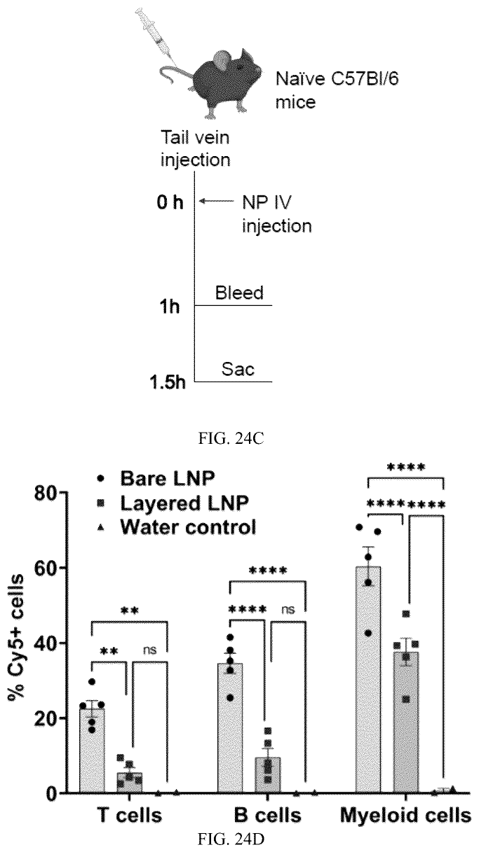

Ab-LLNPs. To improve on-target targeting and uptake of HSPCs, polyanions were further modified with reactive handles to enable attachment of antibodies to improve uptake and targeting (FIG. 18B). PAA-LLNPs were functionalized with antibodies such as anti-CD117 (or anti-cKit), an HSPC-targeting moiety (FIGS. 24A-B). FIG. 24B shows relative to unlayered LNPs, layered and functionalized LNPs showed diameter increases of 45-55 nm and complete charge reversal (−48, −40 mV). Ab-LLNPs were doped with Cy5-conjugated phospholipid to track targeting in vivo (FIGS. 24C-D). C57Bl/6 mice were injected intravenously with either bare or functionalized LNPs. Blood was collected 1 h after injection (FIG. 24C). Cell populations of interest were stained and analyzed for Cy5 uptake. Relative to unlayered LNPs, layered LNPs demonstrated 4.1-, 3.6-, and 1.6-fold reduced uptake in T, B, and myeloid cells (FIG. 24D, demonstrating the importance of the targeted layered system to reduce off-target interactions and improve targeting to HSPCs.

The antibody can be modified with a heterobifunctional linker with a PEG spacer, that reacts on one side to the antibody through, for example, a maleimide-thiol reaction; and with a click linker on the other side, such as azide, TCO, or DBCO, that can react to the modified polyanion on the LLNPs. The modular nature of these systems enables a variety of alternate targeting moieties to be covalently attached, including but not limited to full or fragmented anti-CXCR4, anti-CD45, anti-CD34, or nanobodies against CD117, CXCR4, CD45, and CD34.

Layered and antibody-conjugated LNP intracellular trafficking patterns were assayed via confocal microscopy studies. In mouse lineage-negative cells, optimized mRNA-loaded LNPs achieve >30% transfection at 200 ng/25K cell dosages.

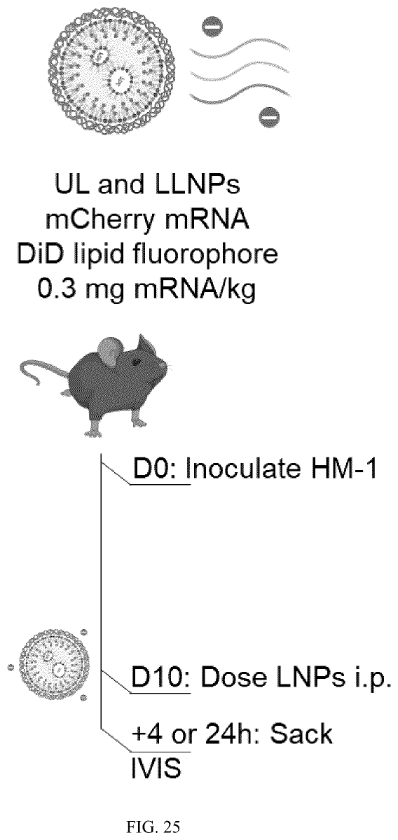

HA and PLE-LLNPs improve accumulation and transfection of ovarian cancer tumor tissue. LNPs containing mCherry-encoding mRNA were layered with each of HA, PLD, and PLE. Furthermore, a fluorescent tag (DiD) was incorporated into NPs to track net accumulation. LNPs were dosed intraperitoneally into mice inoculated with HM-1 ovarian cancer cells 10 days prior. After either 4 or 24 h, mice were sacked and abdominal organs (liver, tumor/omentum tissue, urogenital tract (UGT), and spleen) were analyzed ex vivo for both DiD lipid accumulation and mCherry expression (FIG. 25).

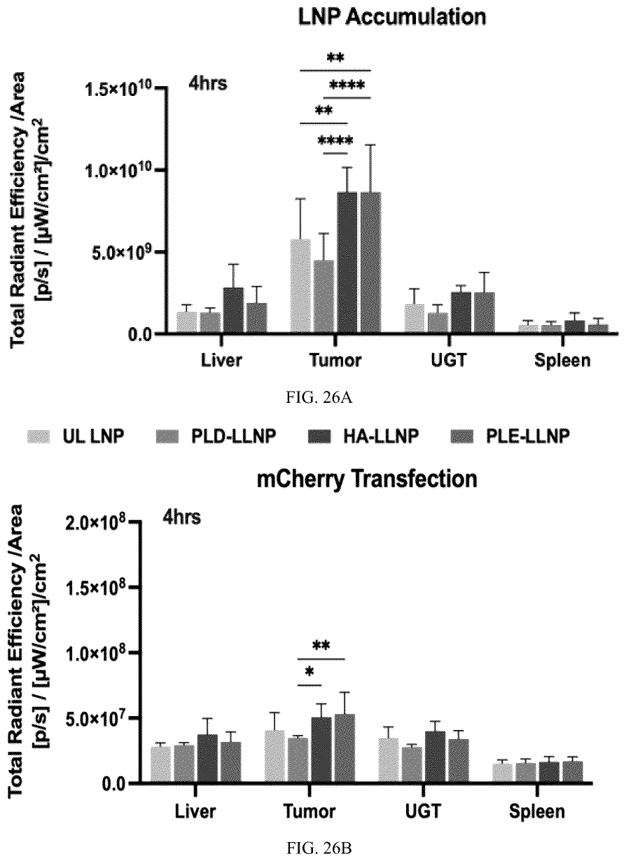

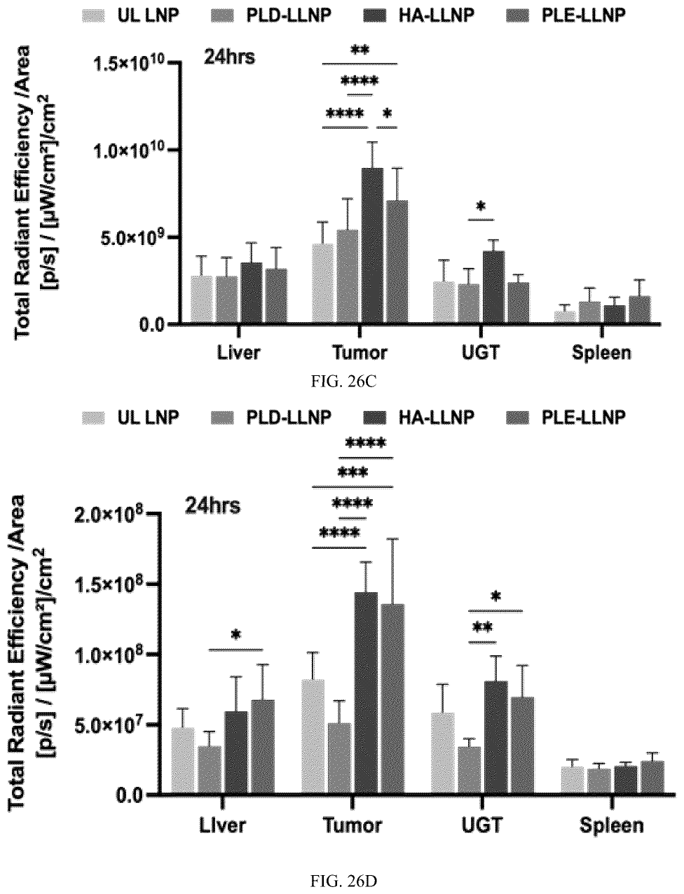

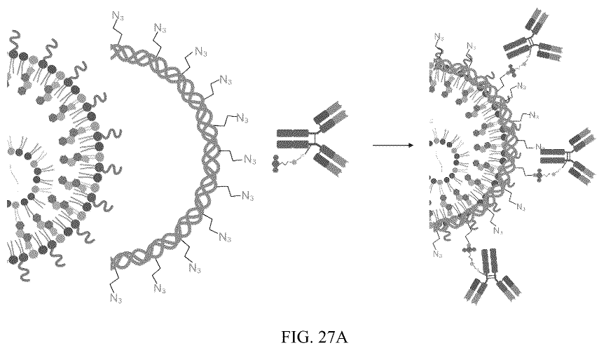

Differences in transfection were observed more prominently 24 h after dosing, in accordance with slower mRNA translation rates (FIGS. 26A-D). Both HA and PLE significantly improved mRNA transfection in tumor tissue over UL LNPs, and to a lesser extent in the urogenital tract (UGT). In terms of accumulation, both HA and PLE-LLNPs significantly improved accumulation in tumor tissue at 4 h and 24 h, over unlayered (UL) particles.

Ab-LLNPs were prepared and characterized by DLS and TEM. LNPs were layered with a polymer mixture of 90% PAA (poly-L-acrylate) and 10% N3-PAA (FIG. 27A). The N3-handles were used to covalently conjugate HSPC-targeting antibodies cKit (CD117) and CD105, as well as a non-targeting isotype (family-matched antibody). Nanoparticle characterization of Ab-LLNPs was performed using DLS (FIG. 27B) and negative-staining room temperature TEM (FIG. 27C). These TEM images show visual confirmation that a range of antibodies can be stably conjugated onto LNP surfaces while maintaining mRNA loading and internal structure (FIG. 27C).

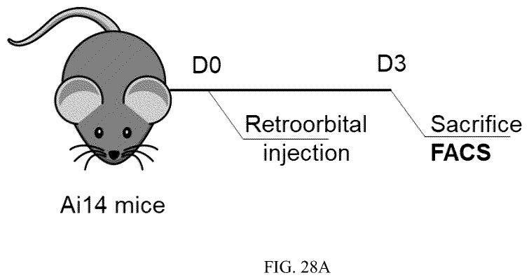

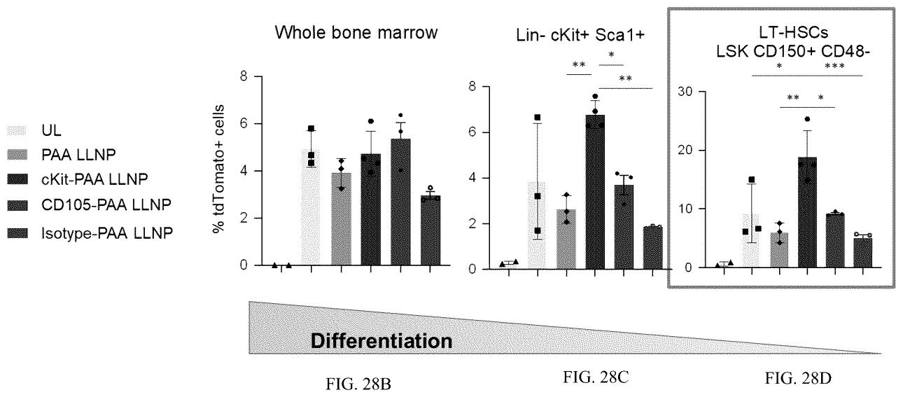

cKit-LLNPs effectively target and transfect HSPCs in vivo. Ab-LLNPs containing Cre mRNA were dosed intravenously in vivo in Cre reporter mice (Ai14), which are genetically engineered to express tdTomato fluorophore in cells transfected with Cre mRNA (FIG. 28A). HSPCs, target cells, reside predominantly in the bone marrow, and make up 0.01% of all bone marrow cells; furthermore, they are difficult to transfect as they are largely quiescent and non-dividing, residing largely outside the cell cycle. cKit-LLNPs exhibited a high degree of specificity towards HSPC transfection in the bone marrow, selectively transfecting significantly more long-term repopulating HSCs (LT-HSCs) than all other LNPs (FIGS. 28B, D). Similar improved transfection was observed in LSK (lineage-negative cells), slightly more differentiated cell subpopulations (FIG. 28C).

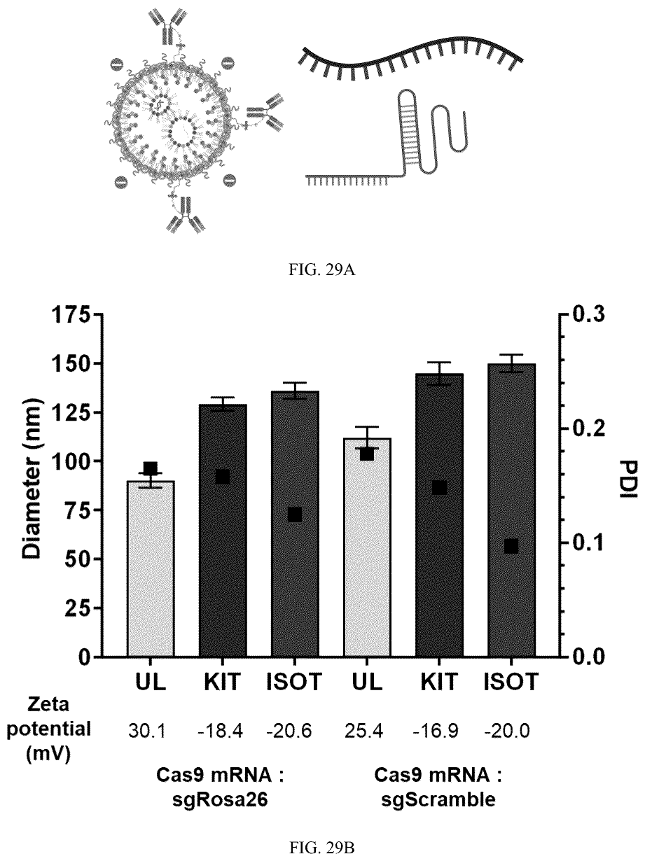

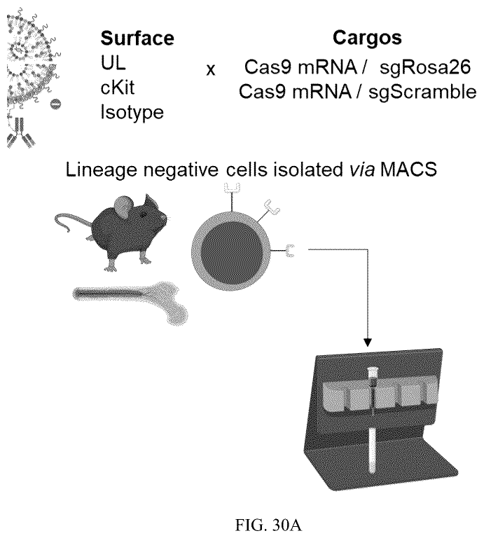

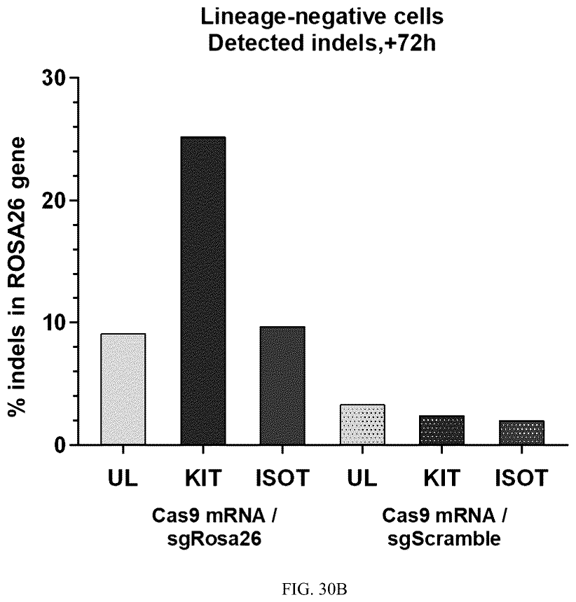

Ab-LLNPs were co-loaded with Cas9 mRNA and guide RNA. PAA-LLNPs conjugated to either cKit or ISOTYPE were alternatively loaded with CRISPR/Cas9 cargos, containing Cas9-encoding mRNA and guide RNAs targeting either model gene Rosa26 or non-targeting scramble (FIGS. 29A-B). The LNPs were evaluated for gene editing potential in lineage negative cells isolated from mouse bone marrow (FIG. 30A). Indels, or Cas9-mediated “cuts” to target gene Rosa26, were evaluated by sequencing genomic DNA of transfected cells (FIG. 30B). After 72 h, cKit-PAA-LLNPs showed 3× improvement in gene editing of lineage-negative cells, over unlayered or isotype-PAA-LLNPs. The targeting appeared highly specific, given that scrambled RNA LNPs induced minimal edits.

LLNPs encapsulating plasmid DNA were modified with the above-optimized surface layer of PAA functionalized with anti-CD117. When injected intravenously into mice, LLNPs demonstrated 4.1-, 3.6-, and 1.6-fold reduced uptake in T, B, and myeloid cells respectively.

Relative to unlayered cationic LNPs, LNPs layered with polyanions exhibited diameter increases of 5 to 10 nanometers and complete surface charge reversal. Encapsulation efficiency of both mRNA and pDNA remained consistent between unlayered and layered LNPs. In vitro studies in EL4 lymphoma cells and ER-HOXB8 progenitor cells demonstrated correlations between lipid molar compositions and LNP transfection. Furthermore, layered LNPs retained transfection capacity, transfecting at comparable or higher rates to unlayered LNPs. LNP surface chemistry can accordingly be modulated to improve targeting and uptake, to aid in the transfection of the bone marrow, among other extrahepatic tissues.

### REFERENCES

1. J. A. Kulkarni, et al., The current landscape of nucleic acid therapeutics. Nat Nanotechnol 16, 630-643 (2021).
2. P. R. Cullis, M. J. Hope, Lipid Nanoparticle Systems for Enabling Gene Therapies. Molecular Therapy 25, 1467-1475 (2017).
3. J. A. Doudna, E. Charpentier, The new frontier of genome engineering with CRISPR-Cas9. Science (1979) 346 (2014).
4. H. Frangoul, et al., CRISPR-Cas9 Gene Editing for Sickle Cell Disease and β-Thalassemia. New England Journal of Medicine 384, 252-260 (2021).
5. G. Schwank, et al., Functional Repair of CFTR by CRISPR/Cas9 in Intestinal Stem Cell Organoids of Cystic Fibrosis Patients. Cell Stem Cell 13, 653-658 (2013).
6. T. Ohmori, et al., CRISPR/Cas9-mediated genome editing via postnatal administration of AAV vector cures haemophilia B mice. Sci Rep 7, 4159 (2017).
7. J. A. Kulkarni, P. R. Cullis, R. Van Der Meel, Lipid Nanoparticles Enabling Gene Therapies: From Concepts to Clinical Utility. Nucleic Acid Ther 28, 146-157 (2018).
8. K. A. Whitehead, R. Langer, D. G. Anderson, Knocking down barriers: advances in siRNA delivery. Nat Rev Drug Discov 8, 129-138 (2009).
9. S. M. Hoy, Patisiran: First Global Approval. Drugs 78, 1625-1631 (2018).
10. E. Samaridou, J. Heyes, P. Lutwyche, Lipid nanoparticles for nucleic acid delivery: Current perspectives. Adv Drug Deliv Rev 154-155, 37-63 (2020).
11. C. Hald Albertsen, et al., The role of lipid components in lipid nanoparticles for vaccines and gene therapy. Adv Drug Deliv Rev 188, 114416 (2022).
12. K. M. Tsoi, et al., Mechanism of hard-nanomaterial clearance by the liver. Nat Mater 15, 1212-1221 (2016).
13. W. Poon, et al., Elimination Pathways of Nanoparticles. ACS Nano 13, 5785-5798 (2019).
14. D. T. Jayaram, S. M. Pustulka, R. G. Mannino, W. A. Lam, C. K. Payne, Protein Corona in Response to Flow: Effect on Protein Concentration and Structure. Biophys J 115, 209-216 (2018).
15. M. Qiu, et al., Lung-selective mRNA delivery of synthetic lipid nanoparticles for the treatment of pulmonary lymphangioleiomyomatosis. PNAS 119 (2022).
16. O. S. Fenton, et al., Synthesis and Biological Evaluation of Ionizable Lipid Materials for the In Vivo Delivery of Messenger RNA to B Lymphocytes. Advanced Materials 29, 1606944 (2017).
17. B. Li, et al., Combinatorial design of nanoparticles for pulmonary mRNA delivery and genome editing. Nat Biotechnol (2023) https:/doi.org/10.1038/s41587-023-01679-x.
18. Q. Cheng, et al., Selective organ targeting (SORT) nanoparticles for tissue-specific mRNA delivery and CRISPR-Cas gene editing. Nat Nanotechnol 15, 313-320 (2020).
19. S. T. LoPresti, M. L. Arral, N. Chaudhary, K. A. Whitehead, The replacement of helper lipids with charged alternatives in lipid nanoparticles facilitates targeted mRNA delivery to the spleen and lungs. Journal of Controlled Release 345, 819-831 (2022).
20. S. W. Morton, Z. Poon, P. T. Hammond, The architecture and biological performance of drug-loaded LbL nanoparticles. Biomaterials 34, 5328-5335 (2013).
21. S. Correa, N. Boehnke, E. Deiss-Yehiely, P. T. Hammond, Solution Conditions Tune and Optimize Loading of Therapeutic Polyelectrolytes into Layer-by-Layer Functionalized Liposomes. ACS Nano 13, 5623-5634 (2019).
22. S. Correa, et al., Tuning Nanoparticle Interactions with Ovarian Cancer through Layer-by-Layer Modification of Surface Chemistry. ACS Nano 14, 2224-2237 (2020).
23. S. Kong, et al., Synergistic combination therapy delivered via layer-by-layer nanoparticles induces solid tumor regression of ovarian cancer. Bioeng Transl Med 8 (2023).
24. K. Y. Choi, et al., Binary Targeting of siRNA to Hematologic Cancer Cells In Vivo Using Layer-by-Layer Nanoparticles. Adv Funct Mater 29, 1900018 (2019).
25. M. Ruponen, et al., Extracellular Glycosaminoglycans Modify Cellular Trafficking of Lipoplexes and Polyplexes. Journal of Biological Chemistry 276, 33875-33880 (2001).
26. Z. J. Deng, et al., Layer-by-Layer Nanoparticles for Systemic Codelivery of an Anticancer Drug and siRNA for Potential Triple-Negative Breast Cancer Treatment. ACS Nano 7, 9571-9584 (2013).
27. K. J. Kauffman, et al., Optimization of Lipid Nanoparticle Formulations for mRNA Delivery in Vivo with Fractional Factorial and Definitive Screening Designs. Nano Lett 15, 7300-7306 (2015).
28. S. C. Semple, et al., Rational design of cationic lipids for siRNA delivery. Nat Biotechnol 28, 172-176 (2010).
29. Y. Dong, et al., Lipopeptide nanoparticles for potent and selective siRNA delivery in rodents and nonhuman primates. Proceedings of the National Academy of Sciences 111, 3955-3960 (2014).
30. J. A. Kulkarni, et al., Design of lipid nanoparticles for in vitro and in vivo delivery of plasmid DNA. Nanomedicine 13, 1377-1387 (2017).
31. E. Deiss-Yehiely, et al., Surface Presentation of Hyaluronic Acid Modulates Nanoparticle-Cell Association. Bioconjug Chem 33, 2065-2075 (2022).
32. E. C. Dreaden, et al., Bimodal Tumor-Targeting from Microenvironment Responsive Hyaluronan Layer-by-Layer (LbL) Nanoparticles. ACS Nano 8, 8374-8382 (2014).
33. P. Patel, N. M. Ibrahim, K. Cheng, The Importance of Apparent pKa in the Development of Nanoparticles Encapsulating siRNA and mRNA. Trends Pharmacol Sci 42, 448-460 (2021).
34. M. J. Carrasco, et al., Ionization and structural properties of mRNA lipid nanoparticles influence expression in intramuscular and intravascular administration. Commun Biol 4, 956 (2021).
35. K. A. Hajj, et al., Branched-Tail Lipid Nanoparticles Potently Deliver mRNA In Vivo due to Enhanced Ionization at Endosomal pH. Small 15, 1805097 (2019).
36. A. Mero, M. Campisi, Hyaluronic Acid Bioconjugates for the Delivery of Bioactive Molecules. Polymers (Basel) 6, 346-369 (2014).
37. M. Chen, V. Gupta, A. C. Anselmo, J. A. Muraski, S. Mitragotri, Topical delivery of hyaluronic acid into skin using SPACE-peptide carriers. Journal of Controlled Release 173, 67-74 (2014).
38. S. A. Dilliard, Q. Cheng, D. J. Siegwart, On the mechanism of tissue-specific mRNA delivery by selective organ targeting nanoparticles. Proceedings of the National Academy of Sciences 118 (2021).
39. A. E. Barberio, et al., Cancer Cell Coating Nanoparticles for Optimal Tumor-Specific Cytokine Delivery. ACS Nano 14, 11238-11253 (2020).
40. C. Mazzaccara, et al., Age-Related Reference Intervals of the Main Biochemical and Hematological Parameters in C57B16J, 129SV/EV and C3H/HeJ Mouse Strains. PLoS One 3, e3772 (2008).
41. G. P. Otto, et al., Clinical Chemistry Reference Intervals for C57BL/6J, C57BL/6N, and C3HeB/FeJ Mice (Mus musculus). J Am Assoc Lab Anim Sci 55, 375-86 (2016).
42. S. Anthiya, et al., Targeted siRNA lipid nanoparticles for the treatment of KRAS-mutant tumors. Journal of Controlled Release 357, 67-83 (2023).
43. Z. R. Cohen, et al., Localized RNAi Therapeutics of Chemoresistant Grade IV Glioma Using Hyaluronan-Grafted Lipid-Based Nanoparticles. ACS Nano 9, 1581-1591 (2015).
44. M. S. Singh, et al., Therapeutic Gene Silencing Using Targeted Lipid Nanoparticles in Metastatic Ovarian Cancer. Small 17, 2100287 (2021).
45. A. Kheirolomoom, et al., In situ T-cell transfection by anti-CD3-conjugated lipid nanoparticles leads to T-cell activation, migration, and phenotypic shift. Biomaterials 281, 121339 (2022).
46. H. Parhiz, et al., PECAM-1 directed re-targeting of exogenous mRNA providing two orders of magnitude enhancement of vascular delivery and expression in lungs independent of apolipoprotein E-mediated uptake. Journal of Controlled Release 291, 106-115 (2018).
47. J. Lesley, Hyaluronan Binding by Cell Surface CD44. Journal of Biological Chemistry (2000) https:/doi.org/10.1074/jbc.M002527200.
48. D. Krejcova, M. Pekarova, B. Safrankova, L. Kubala, The effect of different molecular weight hyaluronan on macrophage physiology. Neuro Endocrinol Lett 30 Suppl 1, 106-11 (2009).
49. N. Boehnke, K. J. Dolph, V. M. Juarez, J. M. Lanoha, P. T. Hammond, Electrostatic Conjugation of Nanoparticle Surfaces with Functional Peptide Motifs. Bioconjug Chem 31, 2211-2219(2020).
50. N. Boehnke, et al., Theranostic Layer-by-Layer Nanoparticles for Simultaneous Tumor Detection and Gene Silencing. Angewandte Chemie 132, 2798-2805 (2020).
51. J. P. Straehla, et al., A predictive microfluidic model of human glioblastoma to assess trafficking of blood-brain barrier-penetrant nanoparticles. Proceedings of the National Academy of Sciences 119 (2022).
52. Kato et al. Nature Reviews Disease Primers 4, 2018.
53. Kumar et al. Chemical Reviews, 121, 11527-11652 (2021).

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

### EMBODIMENTS

Embodiment 1. A lipid nanoparticle (LNP) comprising a core and an outer polyelectrolyte layer comprising a polyelectrolyte compound.

Embodiment 2. The LNP of embodiment 1, wherein the core comprises one or more lipids selected from: an ionizable amino lipid, a phospholipid, a PEG lipid, and a sterol.

Embodiment 3. The LNP of embodiment 1 or 2, wherein the polyelectrolyte compound is a polyanionic compound.

Embodiment 4. The LNP of any one of embodiments 1-3, wherein the polyelectrolyte layer comprises hyaluronic acid (HA), poly-L-glutamic acid (PLE), poly-L-aspartic acid (PLD), or poly acrylic acid (PAA).

Embodiment 5. The LNP of any one of embodiments 1-4, further comprising an agent.

Embodiment 6. The LNP of embodiment 5, wherein the agent is an organic molecule, inorganic molecule, nucleic acid, protein, peptide, polynucleotide, targeting agent, an isotopically labeled chemical compound, vaccine, an immunological agent, or an agent useful in bioprocessing.

Embodiment 7. The LNP of embodiment 6, wherein the agent is a polynucleotide.

Embodiment 8. The LNP of embodiment 7, wherein the polynucleotide is RNA or DNA.

Embodiment 9. The LNP of embodiment 8, wherein the RNA is messenger RNA (mRNA), single stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long noncoding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA

Embodiment 10. The LNP of embodiment 9 wherein the RNA is mRNA.

Embodiment 11. The LNP of embodiment 8 wherein the DNA is plasmid DNA (pDNA).

Embodiment 12. The LNP of any one of embodiments 1-11, wherein the core comprises DSPC, DMG-PEG-2000, cholesterol, and ALC-0315; and the polyelectrolyte layer comprises PLE.

Embodiment 13. The LNP of any one of embodiments 1-11, wherein the core comprises DOPE, DMG-PEG-2000, cholesterol, and an ionizable amino lipid; and the polyelectrolyte layer comprises PLA.

Embodiment 14. The LNP of any one of embodiments 1-13, further comprising a targeting molecule selected from the group consisting of antibodies, antibody fragments, peptides, and nanobodies.

Embodiment 15. The LNP of embodiment 14, wherein an anti-CD117 antibody is covalently conjugated to the polyelectrolyte compound.

Embodiment 16. A method of delivering a polynucleotide to a subject or cell, the method comprising administering to the subject or contacting the cell with an LNP of any one of embodiments 7-15.

Embodiment 17. The method of embodiment 16, wherein the cell is HEK293T or Jurkat Embodiment 18. A method of treating or preventing a disease in a subject in need thereof, the method comprising administering to the subject an LNP of any one of embodiments 1-17.

Embodiment 19. The method of embodiment 18, wherein the disease is a genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease.

Embodiment 20. The method of embodiment 19, wherein the disease is sickle cell anemia.

Embodiment 21. A method of making a LNP comprising an outer polyelectrolyte layer, comprising: providing an LNP core and, contacting the LNP core with the polyelectrolyte with mechanical stirring in the range of 700 RPM to 800 RPM.

1. A nanoparticle, comprising:
(a) a lipid nanoparticle (LNP) core comprising;
an ionizable lipid, wherein the ionizable lipid is positively charged;
a cargo;
a sterol;
a polyethylene glycol (PEG) lipid;
a phospholipid; and
an ionizable lipid; and
(b) an anionic polymer coating the LNP core;
wherein the anionic polymer is not nucleic acid-based.

2. (canceled)

3. The nanoparticle of claim 1, wherein the sterol is cholesterol.

4. The nanoparticle of claim 1, wherein the PEG lipid is DMG-PEG-2000.

5. The nanoparticle of claim 1, wherein the phospholipid is selected from 1,2-dioleoyl-sn-glycero-3-phosphoethanolamine (DOPE), 1,2-distearoyl-sn-glycero-3-phosphocholine (DSPC), and 1,2-dioleoyl-sn-glycero-3-phosphocholine (DOPC).

6. The nanoparticle of claim 1, wherein the ionizable lipid is selected from ALC-0315, DLin-MC3-DMA, DLin-KC2-DMA, cKK-E12, C12-200, and SM-102.

7. The nanoparticle of claim 1, wherein the anionic polymer is carboxylated or sulfonated.

8. The nanoparticle of claim 7, wherein the anionic polymer comprises hyaluronic acid (HA), poly-L-glutamate (PLE), poly-L-aspartate (PLD), polyacrylic acid (PAA), dextran sulfate, chondroitin sulfate, fucoidan, heparin sulfate, alginate, pegylated-poly-L-glutamic acid, pegylated-poly-L-aspartic acid, polysialic acid, carboxymethyl cellulose, methacrylate, sulfated polybeta cyclodextrin.

9-10. (canceled)

11. The nanoparticle of claim 1, wherein the anionic polymer is not DNA or RNA.

12-13. (canceled)

14. The nanoparticle of claim 1, wherein the particle does not comprise a poly-arginine polymer layer between the LNP core and the anionic polymer.

15-19. (canceled)

20. The nanoparticle of claim 1, wherein the cargo is DNA or RNA.

21-24. (canceled)

25. The nanoparticle of claim 1, further comprising a targeting moiety selected from the group consisting of an antibody, nanobody, affibody, aptamer, cyclodextrin, and a derivative or fragment thereof.

26-32. (canceled)

33. The nanoparticle of claim 25, wherein the targeting moiety is an antibody or nanobody selected from the group consisting of anti-CD117, anti-CD105, anti-CD90, anti-CXCR4, anti-CD45, anti-CD4, anti-CD8, anti-CD3, anti-CD19, anti-CD20, and anti-ferritin antibody or nanobody.

34-45. (canceled)

46. A pharmaceutical composition comprising a plurality of nanoparticles of claim 1, and a pharmaceutically acceptable excipient.

47. (canceled)

48. A method of administering a cargo to a subject in need thereof, the method comprising administering to the subject a plurality of nanoparticles of claim 1.

49. A method of delivering a cargo to a target cell, the method comprising contacting the target cell with a nanoparticle of claim 1.

50-54. (canceled)

55. A method of treating a disease in a subject in need thereof, the method comprising administering to the subject a plurality of nanoparticles of claim 1.

56. (canceled)

57. The method of claim 55, wherein the disease is a proliferative disease, an immune disorder, or genetic disease.

58-60. (canceled)

61. A method of editing a gene in a cell, the method comprising contacting the cell with the nanoparticle of claim 1.

62-69. (canceled)

70. A method of reducing non-targeted cell uptake of a nanoparticles, the method comprising coating the nanoparticle with polyacrylic acid (PAA).

71-75. (canceled)

76. A kit comprising:
a plurality of nanoparticles of claim 1; and
instructions for using the plurality of nanoparticles.
